(19) United States
(12) Patent Application Publication
(10) Pub. No.: US 2025/0368714 A1
(43) Pub. Date: Dec. 4, 2025

(54) TREATMENT OF CANCER

(71) Applicant: Immutep S.A.S., SAINT-AUBIN (FR)

(72) Inventor: Frederic TRIEBEL, SAINT-AUBIN (FR)

(21) Appl. No.: 18/837,252

(22) PCT Filed: Feb. 10, 2023
(86) PCT No.: PCT/EP2023/053381

§ 371 Date: Aug. 9, 2024

(30) Foreign Application Priority Data
Feb. 11, 2022 (GB) 2201803.0
Dec. 2, 2022 (GB) 2218181.2

(51) Int. Cl. C07K 14/705; A61K 31/337; A61K 38/00; A61P 35/04

(52) U.S. Cl. CPC C07K 14/70503; A61K 31/337; A61P 35/04; A61K 38/00; C07K 2319/30

(57) ABSTRACT

Use of a LAG-3 protein or derivative thereof for the treatment of a cancer in a subject is described. In certain aspects, the subject has one or more of: a low monocyte count; a Luminal B breast cancer; an age of less than about 85 years; has been previously treated with a CDK4/6 inhibitor; has not previously undergone treatment with a taxane chemotherapy; has an elevated neutrophil to lymphocyte ratio; been diagnosed less than about 5 years ago. In another aspect, the LAG-3 protein or derivative is administered to the subject at a dosage of a molar equivalent of >30 mg to <120 mg of LAG-3 derivative LAG-3Ig fusion protein IMP321. In a further aspect, the LAG-3 protein, or derivative, is administered to the subject on the same day as a chemotherapy agent. In another aspect, the LAG-3 protein, or derivative, is administered to the subject one or more times in the absence of a chemotherapy agent, after one or more dosages of the LAG-3 protein, or derivative, have been administered to the subject before, with, or after one or more dosages of the chemotherapy agent. In a further aspect, the subject is a hormone receptor-positive HER2-neg/low (HR⁺/HER2-neg/low) metastatic breast cancer patient, or a metastatic triple negative breast cancer (TNBC) patient.

### FIELD OF THE INVENTION

This invention relates to the use of a LAG-3 protein or derivative thereof for the treatment of cancer.

### BACKGROUND OF THE INVENTION

Over the past decade, PD-1 and CTLA-4 immune checkpoint inhibitors such as OPDIVO (nivolumab), KEYTRUDA (pembrolizumab) and YERVOY (ipilimumab) have become the standard of care therapies for many forms of cancer, however unfortunately, many patients still fail to respond to these modern medicines. In an effort to improve patient outcomes, significant work has been undertaken to investigate other immune checkpoints, such as LAG-3, TIM-3, VISTA, CD47, IDO and TIGIT. LAG-3 in particular has emerged as a promising checkpoint and a number of companies are developing new inhibitors that target this checkpoint. The aim of a LAG-3 inhibitor, as with the currently approved PD-1 and CTLA-4 inhibitors, is to block the down-regulation of the immune system i.e. taking the “brakes off” the body's immune processes. Significant work has also been undertaken to explore combinations of PD-1 and CTLA-4 immune checkpoint inhibitors with other approved or experimental therapies. Another type of active immunotherapy being investigated are antigen presenting cell (APC) activators. APC activators bind to antigen presenting cells such as dendritic cells, monocytes and macrophages via MHC II molecules. This activates the APCs causing them to become professional antigen presenting cells, thereby presenting antigen to the adaptive immune system. This leads to activation and proliferation of CD4+ (helper) and CD8+ (cytotoxic) T cells. Thus, the aim of APC activators is to “push the gas” on the body's immune system.

Eftilagimod alpha (IMP321 or efti), a soluble dimeric recombinant form of LAG-3, is a first-in-class APC activator under clinical development. By stimulating dendritic cells and other APCs through MHC class II molecules, IMP321 induces a powerful anti-cancer T cell response. IMP321 is described in WO 2009/044273, which also describes the use of IMP321 alone and in combination with a chemotherapy agent for the treatment of cancer. There remains a need in the art for improved cancer therapies and treatment regimens leading to better outcomes for patients. This is especially so for cancers where the prognosis for patients undertaking treatment with current medicines is poor.

Metastatic Breast Cancer (MBC) is a major therapeutic challenge either as a de novo breast cancer diagnosis or as a recurrence after previous treatment of early-stage disease. The aim of MBC treatment is primarily disease control and to palliate symptoms with minimal side effects.

Endocrine therapy (ET) in combination with CDK4/6 inhibitors, other targeted therapies, or alone remains the mainstay in managing hormone receptor positive (HR⁺), HER2-neg/low MBC. However, the likelihood of developing ET resistance is high. Prior to introduction of CDK4/6 inhibitors to the treatment landscape, patients with ET resistance were usually prescribed with single agent chemotherapy with a median overall survival (OS) of ˜24 months. The addition of CDK4/6 inhibitors to ET-based treatment reduced the OS of subsequent chemotherapy with median OS decreasing to ˜12-18 months. There is a high unmet medical need in HR⁺ HER2-neg/low MBC patients who are resistant to endocrine based therapy and are eligible for chemotherapy.

Metastatic triple negative breast cancer (TNBC) is an aggressive disease with poor outcomes. This type of breast cancer is characterized by the lack of expression of estrogen receptor (ER), progesterone receptor (PR) and HER2, and the presence of high histologic grades and mitotic rates. The choice of therapy depends predominantly on PD-L1 expression. For patients with PD-L1-positive tumors (around 40% of all TNBC patients), anti-PD-1 plus chemotherapy is advised. Otherwise, chemotherapy remains the primary systemic treatment, with international guidelines supporting the use of single-agent taxanes (with or without bevacizumab) or anthracyclines as first-line therapy, with median OS up to 18 months. Given the suboptimal outcomes of chemotherapy alone (median OS between 15 to 18 months) or with immune checkpoint inhibitors (ICI; 21 to 25 months) for PD-L1 positive tumors, there is a high unmet medical need for these patients.

### SUMMARY OF THE INVENTION

In one embodiment, the invention relates to a LAG-3 protein, or a derivative thereof that is able to bind to MHC class II molecules, for use in preventing, treating, or ameliorating a cancer in a subject with a low monocyte count.

In another embodiment, the invention relates to the use of a LAG-3 protein, or a derivative thereof that is able to bind to MHC class II molecules, in the manufacture of a medicament for the prevention, treatment, or amelioration of a cancer in a subject with a low monocyte count.

In yet another embodiment, the invention relates to the use of a LAG-3 protein, or a derivative thereof that is able to bind to MHC class II molecules, for the prevention, treatment, or amelioration of a cancer in a subject with a low monocyte count.

In a further embodiment, the invention provides a method of preventing, treating, or ameliorating a cancer in a subject with a low monocyte count, the method comprising administering to the subject in need of such prevention, treatment, or amelioration a LAG-3 protein, or a derivative thereof that is able to bind to MHC class II molecules.

In one embodiment, the invention relates to a LAG-3 protein, or a derivative thereof that is able to bind to MHC class II molecules, for use in preventing, treating, or ameliorating a Luminal B breast cancer in a subject.

In another embodiment, the invention relates to the use of a LAG-3 protein, or a derivative thereof that is able to bind to MHC class II molecules, in the manufacture of a medicament for the prevention, treatment, or amelioration of a Luminal B breast cancer in a subject.

In yet another embodiment, the invention relates to the use of a LAG-3 protein, or a derivative thereof that is able to bind to MHC class II molecules, for the prevention, treatment, or amelioration of a Luminal B breast cancer in a subject.

In a further embodiment, the invention provides a method of preventing, treating, or ameliorating a Luminal B breast cancer, the method comprising administering to a subject in need of such prevention, treatment, or amelioration a LAG-3 protein, or a derivative thereof that is able to bind to MHC class II molecules.

In one embodiment, the invention relates to a LAG-3 protein, or a derivative thereof that is able to bind to MHC class II molecules, for use in preventing, treating, or ameliorating a cancer in a subject with an age of less than about 85 years.

In another embodiment, the invention relates to the use of a LAG-3 protein, or a derivative thereof that is able to bind to MHC class II molecules, in the manufacture of a medicament for the prevention, treatment, or amelioration of a cancer in a subject with an age of less than about 85 years.

In yet another embodiment, the invention relates to the use of a LAG-3 protein, or a derivative thereof that is able to bind to MHC class II molecules, for the prevention, treatment, or amelioration of a cancer in a subject with an age of less than about 85 years.

In a further embodiment, the invention provides a method of preventing, treating, or ameliorating a cancer in a subject with an age of less than about 85 years, the method comprising administering to the subject in need of such prevention, treatment, or amelioration a LAG-3 protein, or a derivative thereof that is able to bind to MHC class II molecules.

In one embodiment, the invention relates to a LAG-3 protein, or a derivative thereof that is able to bind to MHC class II molecules, for use in preventing, treating, or ameliorating a cancer in a subject that has been previously treated with a CDK4/6 inhibitor.

In another embodiment, the invention relates to the use of a LAG-3 protein, or a derivative thereof that is able to bind to MHC class II molecules, in the manufacture of a medicament for the prevention, treatment, or amelioration of a cancer in a subject that has been previously treated with a CDK4/6 inhibitor.

In yet another embodiment, the invention relates to the use of a LAG-3 protein, or a derivative thereof that is able to bind to MHC class II molecules, for the prevention, treatment, or amelioration of a cancer in a subject that has been previously treated with a CDK4/6 inhibitor.

In a further embodiment, the invention provides a method of preventing, treating, or ameliorating a cancer in a subject that has been previously treated with a CDK4/6 inhibitor, the method comprising administering to the subject in need of such prevention, treatment, or amelioration a LAG-3 protein, or a derivative thereof that is able to bind to MHC class II molecules.

In one embodiment, the invention relates to a LAG-3 protein, or a derivative thereof that is able to bind to MHC class II molecules, for use in preventing, treating, or ameliorating a cancer in a subject that has not previously undergone treatment with a taxane chemotherapy.

In another embodiment, the invention relates to the use of a LAG-3 protein, or a derivative thereof that is able to bind to MHC class II molecules, in the manufacture of a medicament for the prevention, treatment, or amelioration of a cancer in a subject that has not previously undergone treatment with a taxane chemotherapy.

In yet another embodiment, the invention relates to the use of a LAG-3 protein, or a derivative thereof that is able to bind to MHC class II molecules, for the prevention, treatment, or amelioration of a cancer in a subject that has not previously undergone treatment with a taxane chemotherapy.

In a further embodiment, the invention provides a method of preventing, treating, or ameliorating a cancer in a subject that has not previously undergone treatment with a taxane chemotherapy, the method comprising administering to the subject in need of such prevention, treatment, or amelioration a LAG-3 protein, or a derivative thereof that is able to bind to MHC class II molecules.

In one embodiment, the invention relates to a LAG-3 protein, or a derivative thereof that is able to bind to MHC class II molecules, for use in preventing, treating, or ameliorating a cancer in a subject that has an elevated neutrophil to lymphocyte ratio.

In another embodiment, the invention relates to the use of a LAG-3 protein, or a derivative thereof that is able to bind to MHC class II molecules, in the manufacture of a medicament for the prevention, treatment, or amelioration of a cancer in a subject that has an elevated neutrophil to lymphocyte ratio.

In yet another embodiment, the invention relates to the use of a LAG-3 protein, or a derivative thereof that is able to bind to MHC class II molecules, for the prevention, treatment, or amelioration of a cancer in a subject that has an elevated neutrophil to lymphocyte ratio.

In a further embodiment, the invention provides a method of preventing, treating, or ameliorating a cancer in a subject that has an elevated neutrophil to lymphocyte ratio, the method comprising administering to the subject in need of such prevention, treatment, or amelioration a LAG-3 protein, or a derivative thereof that is able to bind to MHC class II molecules.

In one embodiment, the invention relates to a LAG-3 protein, or a derivative thereof that is able to bind to MHC class II molecules, for use in preventing, treating, or ameliorating a cancer in a subject diagnosed less than about 5 years ago.

In another embodiment, the invention relates to the use of a LAG-3 protein, or a derivative thereof that is able to bind to MHC class II molecules, in the manufacture of a medicament for the prevention, treatment, or amelioration of a cancer in a subject diagnosed less than about 5 years ago.

In yet another embodiment, the invention relates to the use of a LAG-3 protein, or a derivative thereof that is able to bind to MHC class II molecules, for the prevention, treatment, or amelioration of a cancer in a subject diagnosed less than about 5 years ago.

In a further embodiment, the invention provides a method of preventing, treating, or ameliorating a cancer in a subject diagnosed less than about 5 years ago, the method comprising administering to the subject in need of such prevention, treatment, or amelioration a LAG-3 protein, or a derivative thereof that is able to bind to MHC class II molecules.

In a further embodiment, the invention provides a LAG-3 protein, or a derivative thereof that is able to bind to MHC class II molecules, for use in preventing, treating, or ameliorating a cancer in a subject, wherein the LAG-3 protein, or derivative, is to be administered to the subject at a dosage of a molar equivalent of >30 mg to <200 mg of LAG-3 derivative LAG-3Ig fusion protein IMP321.

In a further embodiment, the invention provides a LAG-3 protein, or a derivative thereof that is able to bind to MHC class II molecules, for use in preventing, treating, or ameliorating a cancer in a subject, wherein the LAG-3 protein, or derivative, is to be administered to the subject at a dosage of a molar equivalent of >30 mg to <120 mg of LAG-3 derivative LAG-3Ig fusion protein IMP321.

In a further embodiment, the invention provides use of a LAG-3 protein, or a derivative thereof that is able to bind to MHC class II molecules, in the manufacture of a medicament for preventing, treating, or ameliorating a cancer in a subject, wherein the LAG-3 protein, or derivative, is to be administered to the subject at a dosage of a molar equivalent of >30 mg to <200 mg of LAG-3 derivative LAG-3Ig fusion protein IMP321.

In a further embodiment, the invention provides use of a LAG-3 protein, or a derivative thereof that is able to bind to MHC class II molecules, in the manufacture of a medicament for preventing, treating, or ameliorating a cancer in a subject, wherein the LAG-3 protein, or derivative, is to be administered to the subject at a dosage of a molar equivalent of >30 mg to <120 mg of LAG-3 derivative LAG-3Ig fusion protein IMP321.

In a further embodiment, the invention provides a method of preventing, treating, or ameliorating a cancer in a subject, the method comprising administering to the subject a LAG-3 protein, or a derivative thereof that is able to bind to MHC class II molecules, at a dosage of a molar equivalent of >30 mg to <200 mg of LAG-3 derivative LAG-3Ig fusion protein IMP321.

In a further embodiment, the invention provides a method of preventing, treating, or ameliorating a cancer in a subject, the method comprising administering to the subject a LAG-3 protein, or a derivative thereof that is able to bind to MHC class II molecules, at a dosage of a molar equivalent of >30 mg to <120 mg of LAG-3 derivative LAG-3Ig fusion protein IMP321.

In a further embodiment, the invention provides a LAG-3 protein, or a derivative thereof that is able to bind to MHC class II molecules, for use in preventing, treating, or ameliorating a cancer in a subject, wherein the LAG-3 protein, or derivative, is to be administered to the subject on the same day as a chemotherapy agent.

In a further embodiment, the invention provides use of a LAG-3 protein, or a derivative thereof that is able to bind to MHC class II molecules, in the manufacture of a medicament for preventing, treating, or ameliorating a cancer in a subject, wherein the LAG-3 protein, or derivative, is to be administered to the subject on the same day as a chemotherapy agent.

In a further embodiment, the invention provides a method of preventing, treating, or ameliorating a cancer in a subject, the method comprising administering to the subject a LAG-3 protein, or a derivative thereof that is able to bind to MHC class II molecules, on the same day as a chemotherapy agent.

Optionally, the LAG-3 protein, or derivative, is to be administered to the subject on Day 1 and Day 15 of a four-week cycle, and the chemotherapy agent is to be administered to the subject on Day 1, Day 8, and Day 15 of the four-week cycle, optionally wherein the four-week cycle is repeated for four to eight cycles, preferably six cycles.

In a further embodiment, the invention provides a LAG-3 protein, or a derivative thereof that is able to bind to MHC class II molecules, for use in preventing, treating, or ameliorating a cancer in a subject, wherein the LAG-3 protein, or derivative, is to be administered to the subject one or more times in the absence of a chemotherapy agent, after one or more dosages of the LAG-3 protein, or derivative, have been administered to the subject before, with, or after one or more dosages of the chemotherapy agent.

In a further embodiment, the invention provides use of a LAG-3 protein, or a derivative thereof that is able to bind to MHC class II molecules, in the manufacture of a medicament for preventing, treating, or ameliorating a cancer in a subject, wherein the LAG-3 protein, or derivative, is to be administered to the subject one or more times in the absence of a chemotherapy agent, after one or more dosages of the LAG-3 protein, or derivative, have been administered to the subject before, with, or after one or more dosages of the chemotherapy agent.

In a further embodiment, the invention provides a method of preventing, treating, or ameliorating a cancer in a subject, the method comprising administering to the subject a LAG-3 protein, or a derivative thereof that is able to bind to MHC class II molecules, one or more times in the absence of a chemotherapy agent, after one or more dosages of the LAG-3 protein, or derivative, have been administered to the subject before, with, or after one or more dosages of the chemotherapy agent.

In a further embodiment, the invention provides a LAG-3 protein, or a derivative thereof that is able to bind to MHC class II molecules, for use in preventing, treating, or ameliorating a metastatic breast cancer in a subject, wherein the subject is a hormone receptor-positive HER2-neg/low (HR⁺/HER2-neg/low) metastatic breast cancer patient, or a metastatic triple negative breast cancer (TNBC) patient.

In a further embodiment, the invention provides use of a LAG-3 protein, or a derivative thereof that is able to bind to MHC class II molecules, in the manufacture of a medicament for preventing, treating, or ameliorating a metastatic breast cancer in a subject, wherein the subject is a hormone receptor-positive HER2-neg/low (HR⁺/HER2-neg/low) metastatic breast cancer patient, or a metastatic triple negative breast cancer (TNBC) patient.

In a further embodiment, the invention provides a method of preventing, treating, or ameliorating a metastatic breast cancer in a subject, the method comprising administering to the subject a LAG-3 protein, or a derivative thereof that is able to bind to MHC class II molecules, wherein the subject is a hormone receptor-positive HER2-neg/low (HR⁺/HER2-neg/low) metastatic breast cancer patient, or a metastatic triple negative breast cancer (TNBC) patient.

### BRIEF DESCRIPTION OF THE DRAWINGS

FIG. 1 shows amino acid sequence of mature human LAG-3 protein (SEQ ID NO:1). The four extracellular Ig superfamily domains are at amino acid residues: 1-149 (D1); 150-239 (D2); 240-330 (D3); and 331-412 (D4). The amino acid sequence of the extra-loop structure of the D1 domain of human LAG-3 protein is shown underlined in bold (SEQ ID NO:2);

FIG. 2 depicts the main Breast Cancer subtypes by HR and HER2 status (Source: Administration, F. a. D. 2022. FDA Approves First Targeted Therapy for HER2-Low Breast Cancer FDA.pdf);

FIG. 3 shows HR⁺ HER2-neg/low MBC Treatment Algorithm (Source: Adapted from Borges, V. F. 2021. Options for Endocrine-Refractory, Hormone Receptor-Positive Breast Cancer: Which Target and When? American Society of Clinical Oncology);

FIG. 4: Summarized algorithm for treatment of mTNBC based on ESMO guidelines;

FIG. 5 shows the AIPAC-003 trial flow chart;

FIG. 6 shows the algorithm to define the optimal biological dose;

FIG. 7 shows Kaplan-Meier Curve for Overall Survival Probability for Patients with BOR of CR/PR (red) and BOR of PD+SD-NA (blue). BOR was assessed by RECIST 1.1. Exploratory Analysis from AIPAC;

FIG. 8 shows pre- and post-treatment CD8⁺ T cells Counts (left) and Correlation with Overall Survival (right); AIPAC study (Source: (Wildiers, et al. 2021. Final results from AIPAC: A phase IIb trial comparing eftilagimod alpha (soluble LAG-3 protein) in combination with weekly paclitaxel in HR+ HER2-MBC. Poster presented at SITC 2021);

FIG. 9 shows change of absolute lymphocyte count (ALC) from baseline (left) and correlated with overall survival (right); and

FIG. 10 shows a schematic overview of the AIPAC-003 trial.

### DETAILED DESCRIPTION OF THE INVENTION

Methods of Treatment in Patient Subgroup with Low Starting Monocyte Count

In one embodiment, the invention relates to a LAG-3 protein, or a derivative thereof that is able to bind to MHC class II molecules, for use in preventing, treating, or ameliorating a cancer in a subject with a low monocyte count.

In another embodiment, the invention relates to the use of a LAG-3 protein, or a derivative thereof that is able to bind to MHC class II molecules, in the manufacture of a medicament for the prevention, treatment, or amelioration of a cancer in a subject with a low monocyte count.

In yet another embodiment, the invention relates to the use of a LAG-3 protein, or a derivative thereof that is able to bind to MHC class II molecules, for the prevention, treatment, or amelioration of a cancer in a subject with a low monocyte count.

In a further embodiment, the invention provides a method of preventing, treating, or ameliorating a cancer in a subject with a low monocyte count, the method comprising administering to the subject in need of such prevention, treatment, or amelioration a LAG-3 protein, or a derivative thereof that is able to bind to MHC class II molecules.

Exemplary cancers that may be treated according to the invention include, but are not limited to, breast cancer, skin cancer, lung cancer (especially NSCLC), ovarian cancer, renal cancer, colon cancer, colorectal cancer, gastric cancer, esophageal cancer, pancreatic cancer, bladder cancer, urothelial cancer, liver cancer, melanoma (for example, metastatic malignant melanoma), prostate cancer (for example hormone refractory prostate adenocarcinoma), head and neck cancer (for example, head and neck squamous cell carcinoma), cervical cancer, thyroid cancer, glioblastoma, glioma, leukemia, lymphoma (for example, a B cell lymphoma), adrenal gland cancer, AIDS-associated cancer, alveolar soft part sarcoma, astrocytic tumor, bone cancer, brain and spinal cord cancer, metastatic brain tumor, carotid body tumor, chondrosarcoma, chordoma, chromophobe renal cell carcinoma, clear cell carcinoma, cutaneous benign fibrous histiocytoma, desmoplastic small round cell tumor, ependymoma, Ewing's tumor, extraskeletal myxoid chondrosarcoma, fibrogenesis imperfecta ossium, fibrous dysplasia of the bone, gallbladder or bile duct cancer, gestational trophoblastic disease, germ cell tumor, haematological malignancy, hepatocellular carcinoma, islet cell tumor, Kaposi's sarcoma, kidney cancer, lipoma/benign lipomatous tumor, liposarcoma/malignant lipomatous tumor, medulloblastoma, meningioma, Merkel cell carcinoma, multiple endocrine neoplasia, multiple myeloma, myelodysplasia syndrome, neuroblastoma, neuroendocrine tumor, papillary thyroid carcinoma, parathyroid tumor, pediatric cancer, peripheral nerve sheath tumor, phaeochromocytoma, pituitary tumor, prostate cancer, posterior uveal melanoma, rare hematologic disorder, renal metastatic cancer, rhabdoid tumor, rhabdomyosarcoma, sarcoma, soft-tissue sarcoma, squamous cell cancer, stomach cancer, synovial sarcoma, testicular cancer, thymic carcinoma, thymoma, thyroid metastatic cancer, and uterine cancer.

In one embodiment, the cancer is a breast cancer. Suitably, the breast cancer is an adenocarcinoma of the breast.

According to embodiments of the invention, the cancer may have progressed to metastatic disease.

In another embodiment, the breast cancer is a hormone receptor-positive cancer (estrogen-receptor positive and/or progesterone-receptor positive), which may be HER2 positive or HER2 negative. In one embodiment, the hormone receptor-positive cancer is HER2 negative. The hormone receptor-positive cancer may be a hormone receptor-positive metastatic breast cancer. In an embodiment, the hormone receptor-positive cancer is hormone receptor-positive HER2 negative metastatic breast cancer.

In an embodiment, the hormone receptor-positive cancer is a Luminal B breast cancer. Luminal B breast cancer is hormone-receptor positive, and either HER2 positive or HER2 negative and has high levels of Ki-67. Luminal B cancers generally grow slightly faster than Luminal A cancers and their prognosis is slightly worse.

In one embodiment, the hormone receptor-positive cancer is HER2 negative and is the Luminal B sub-type. As with other embodiments of the invention, the hormone receptor-positive HER2 negative breast cancer with the Luminal B sub-type may have progressed to metastatic disease. Thus, in an embodiment, the hormone receptor-positive cancer is hormone receptor-positive HER2 negative metastatic breast cancer with the Luminal B sub-type.

In another embodiment, the hormone receptor-positive cancer is a Luminal A breast cancer. Luminal A breast cancer is hormone-receptor positive (estrogen-receptor positive and/or progesterone-receptor positive), HER2 negative, and has low levels of the protein Ki-67.

In yet another embodiment, the breast cancer is triple negative breast cancer (estrogen-receptor negative, progesterone-receptor negative and HER2 negative).

In one particular embodiment, the breast cancer is hormone receptor-positive HER2-neg/low (HR⁺/HER2-neg/low) breast cancer, or triple negative breast cancer (TNBC).

Suitably, the breast cancer is hormone receptor-positive HER2-neg/low (HR⁺/HER2-neg/low) metastatic breast cancer, or metastatic triple negative breast cancer (TNBC).

In a further embodiment, the breast cancer is HER2-enriched breast cancer. HER2-enriched breast cancer is hormone-receptor negative (estrogen-receptor negative and progesterone-receptor negative) and HER2 positive. HER2-enriched cancers tend to grow faster than luminal cancers and can have a worse prognosis.

In some embodiments, the LAG-3 protein or derivative thereof is administered parenterally (including by subcutaneous, intravenous, or intramuscular injection). In particular embodiments, the LAG-3 protein or derivative thereof is administered subcutaneously by injection.

According to certain embodiments of the invention, patients with a low starting monocyte count are selected for treatment. As defined herein, a “low monocyte count” is less than about 0.25×10⁹ cells/L of blood at baseline. “Baseline” means prior to commencement of treatment according to the invention.

### Methods of Treatment in Luminal B Breast Cancer Subgroup

In one embodiment, the invention relates to a LAG-3 protein, or a derivative thereof that is able to bind to MHC class II molecules, for use in preventing, treating, or ameliorating a Luminal B breast cancer in a subject.

In another embodiment, the invention relates to the use of a LAG-3 protein, or a derivative thereof that is able to bind to MHC class II molecules, in the manufacture of a medicament for the prevention, treatment, or amelioration of a Luminal B breast cancer in a subject.

In yet another embodiment, the invention relates to the use of a LAG-3 protein, or a derivative thereof that is able to bind to MHC class II molecules, for the prevention, treatment, or amelioration of a Luminal B breast cancer in a subject.

In a further embodiment, the invention provides a method of preventing, treating, or ameliorating a Luminal B breast cancer in a subject, the method comprising administering to the subject in need of such prevention, treatment, or amelioration a LAG-3 protein, or a derivative thereof that is able to bind to MHC class II molecules.

As explained herein, a Luminal B breast cancer is hormone-receptor positive, and either HER2 positive or HER2 negative and has high levels of Ki-67. Luminal B cancers generally grow slightly faster than Luminal A cancers and their prognosis is slightly worse.

In one embodiment, the Luminal B breast cancer is HER2 negative. In another embodiment, the Luminal B breast cancer is HER2 negative, and has progressed to metastatic disease. Thus, in the embodiments of the invention, the Luminal B breast cancer is HER2 negative metastatic breast cancer.

In one particular embodiment, the Luminal B breast cancer is hormone receptor-positive HER2-neg/low breast cancer. Suitably, the Luminal B breast cancer is hormone receptor-positive HER2 neg/low metastatic breast cancer.

In another embodiment, the Luminal B breast cancer is HER2 positive. In another embodiment, the Luminal B breast cancer is HER2 positive, and has progressed to metastatic disease. Thus, in the embodiments of the invention, the Luminal B breast cancer is HER2 positive metastatic breast cancer.

In yet another embodiment, the subject has a Luminal B breast cancer and the subject also has a low monocyte count.

In an embodiment, the subject has a Luminal B breast cancer which is HER2 negative and the subject also has a low monocyte count. In another embodiment, the subject has a Luminal B breast cancer which is HER2 neg/low and the subject also has a low monocyte count.

Alternatively, the subject has a Luminal B breast cancer which is HER2 positive and the subject also has a low monocyte count.

In one particular embodiment, the subject has metastatic Luminal B breast cancer which is HER2 negative and the subject also has a low monocyte count. In another embodiment, the subject has a metastatic Luminal B breast cancer which is HER2 neg/low and the subject also has a low monocyte count.

### Methods of Treatment in Age Based Patient Subgroup

In one embodiment, the invention relates to a LAG-3 protein, or a derivative thereof that is able to bind to MHC class II molecules, for use in preventing, treating, or ameliorating a cancer in a subject with an age of less than about 85 years.

In another embodiment, the invention relates to the use of a LAG-3 protein, or a derivative thereof that is able to bind to MHC class II molecules, in the manufacture of a medicament for the prevention, treatment, or amelioration of a cancer in a subject with an age of less than about 85 years.

In yet another embodiment, the invention relates to the use of a LAG-3 protein, or a derivative thereof that is able to bind to MHC class II molecules, for the prevention, treatment, or amelioration of a cancer in a subject with an age of less than about 85 years.

In a further embodiment, the invention provides a method of preventing, treating, or ameliorating a cancer in a subject with an age of less than about 85 years, the method comprising administering to the subject in need of such prevention, treatment, or amelioration a LAG-3 protein, or a derivative thereof that is able to bind to MHC class II molecules.

Suitably, the subject has an age of less than about 85 years, less than about 80 years, less than about 75 years, less than about 70 years, less than about 65 years, less than about 60 years, less than about 55 years, less than about 50 years, less than about 45 years, or less than about 40 years.

In one embodiment, the subject has an age between about 18 years and about 85 years. In another embodiment, the subject has an age between about 18 years and about 80 years. In yet another embodiment, the subject has an age between about 18 years and about 75 years. In a further embodiment, the subject has an age between about 18 years and about 70 years. In yet a further embodiment, the subject has an age between about 18 years and about 65 years. In an embodiment, the subject has an age between about 18 years and about 60 years. In another embodiment, the subject has an age between about 18 years and about 55 years. In yet another embodiment, the subject has an age between about 18 years and about 50 years. In a further embodiment, the subject has an age between about 18 years and about 45 years. In yet a further embodiment, the subject has an age between about 18 years and about 40 years.

In another embodiment, the subject is pre-menopausal.

Suitably, the subject has an age of less than about 85 years, less than about 84 years, less than about 83 years, less than about 82 years, less than about 81 years, less than about 80 years, less than about 79 years, less than about 78 years, less than about 77 years, less than about 76 years, less than about 75 years, less than about 74 years, less than about 73 years, less than about 72 years, less than about 71 years, less than about 70 years, less than about 69 years, less than about 68 years, less than about 67 years, less than about 66 years, less than about 65 years, less than about 64 years, less than about 63 years, less than about 62 years, less than about 61 years, less than about 60 years, less than about 59 years, less than about 58 years, less than about 57 years, less than about 56 years, or less than about 55 years, less than about 54 years, less than about 53 years, less than about 52 years, less than about 51 years, less than about 50 years, less than about 49 years, less than about 48 years, less than about 47 years, less than about 46 years, less than about 45 years, less than about 44 years, less than about 43 years, less than about 42 years, less than about 41 years, or less than about 40 years.

In each case, the patient may optionally be older than about 18 years and less than the age recited herein.

In one particular embodiment, the subject has an age of less than about 65 years.

In another particular embodiment, the subject has an age of less than about 53 years.

Exemplary cancers that may be treated according to this embodiment of the invention include, but are not limited to, those that are described hereinabove.

In one particular embodiment, the cancer is breast cancer. Suitably, the breast cancer is hormone receptor-positive HER2-neg/low (HR⁺/HER2-neg/low) breast cancer, or triple negative breast cancer (TNBC).

Suitably, the breast cancer is hormone receptor-positive HER2-neg/low (HR⁺/HER2-neg/low) metastatic breast cancer, or metastatic triple negative breast cancer (TNBC).

Methods of Treatment in Patient Subgroup Previously Treated with a CDK4/6 Inhibitor

In one embodiment, the invention relates to a LAG-3 protein, or a derivative thereof that is able to bind to MHC class II molecules, for use in preventing, treating, or ameliorating a cancer in a subject that has been previously treated with a CDK4/6 inhibitor.

In another embodiment, the invention relates to the use of a LAG-3 protein, or a derivative thereof that is able to bind to MHC class II molecules, in the manufacture of a medicament for the prevention, treatment, or amelioration of a cancer in a subject that has been previously treated with a CDK4/6 inhibitor.

In yet another embodiment, the invention relates to the use of a LAG-3 protein, or a derivative thereof that is able to bind to MHC class II molecules, for the prevention, treatment, or amelioration of a cancer in a subject that has been previously treated with a CDK4/6 inhibitor.

In a further embodiment, the invention provides a method of preventing, treating, or ameliorating a cancer in a subject that has been previously treated with a CDK4/6 inhibitor, the method comprising administering to the subject in need of such prevention, treatment, or amelioration a LAG-3 protein, or a derivative thereof that is able to bind to MHC class II molecules.

CDK4/6 inhibitors are a new class of treatments for cancer, especially hormone receptor positive HER2 negative metastatic breast cancer, that target cyclin-dependent kinase 4 and 6. Exemplary CDK4/6 inhibitors include, but are not limited to, palbociclib, ribociclib and abemaciclib.

Suitably, in an embodiment of the invention, the subject has previously undertaken therapy with a CDK4/6 inhibitor, but their disease has continued to progress and they require an alternative treatment option.

Exemplary cancers that may be treated according to this embodiment of the invention include, but are not limited to, those that are described hereinabove.

In one embodiment, the cancer is a breast cancer. In another embodiment, the breast cancer is hormone receptor positive HER2 negative (HR⁺/HER2−) breast cancer or hormone receptor-positive HER2-neg/low (HR⁺/HER2-neg/low) breast cancer.

Suitably, the breast cancer is hormone receptor-positive HER2-neg/low (HR⁺/HER2-neg/low) breast cancer.

In yet another embodiment, the hormone receptor positive HER2 negative breast cancer is hormone receptor positive HER2 negative metastatic breast cancer or hormone receptor-positive HER2-neg/low (HR⁺/HER2-neg/low) metastatic breast cancer.

Suitably, the breast cancer is hormone receptor-positive HER2-neg/low (HR⁺/HER2-neg/low) metastatic breast cancer.

Methods of Treatment in Patient Subgroup not Previously Treated with Taxane Therapy

In one embodiment, the invention relates to a LAG-3 protein, or a derivative thereof that is able to bind to MHC class II molecules, for use in preventing, treating, or ameliorating a cancer in a subject that has not previously undergone treatment with a taxane chemotherapy.

In another embodiment, the invention relates to the use of a LAG-3 protein, or a derivative thereof that is able to bind to MHC class II molecules, in the manufacture of a medicament for the prevention, treatment, or amelioration of a cancer in a subject that has not previously undergone treatment with a taxane chemotherapy.

In yet another embodiment, the invention relates to the use of a LAG-3 protein, or a derivative thereof that is able to bind to MHC class II molecules, for the prevention, treatment, or amelioration of a cancer in a subject that has not previously undergone treatment with a taxane chemotherapy.

In a further embodiment, the invention provides a method of preventing, treating, or ameliorating a cancer in a subject that has not previously undergone treatment with a taxane chemotherapy, the method comprising administering to the subject in need of such prevention, treatment, or amelioration a LAG-3 protein, or a derivative thereof that is able to bind to MHC class II molecules.

Suitably, in an embodiment of the invention, the subject has not previously undergone treatment with a taxane chemotherapy. Taxane chemotherapy agents feature a taxadiene structure, and act by binding to tubulin, thus stabilizing the microtubule polymer and protecting it from disassembly. This in turn blocks the progression of mitosis, triggering apoptosis (cell death). Taxane chemotherapies are effective in a wide variety of cancers including breast, ovarian, lung, pancreatic, prostate, and head and neck cancers.

Exemplary taxane chemotherapies include, but are not limited to, paclitaxel, nab-paclitaxel, docetaxel, cabazitaxel, larotaxel, milataxel, ortataxel, taxoprexin, opaxio, tesetaxel, and BMS-184476.

Exemplary cancers that may be treated according to this embodiment of the invention include, but are not limited to, those that are described hereinabove.

In one particular embodiment, the cancer is breast cancer. Suitably, the breast cancer is hormone receptor-positive HER2-neg/low (HR⁺/HER2-neg/low) breast cancer, or triple negative breast cancer (TNBC).

Suitably, the breast cancer is hormone receptor-positive HER2-neg/low (HR⁺/HER2-neg/low) metastatic breast cancer, or metastatic triple negative breast cancer (TNBC).

Methods of Treatment in Patient Subgroup Having an Elevated Neutrophil to Lymphocyte Ratio

In one embodiment, the invention relates to a LAG-3 protein, or a derivative thereof that is able to bind to MHC class II molecules, for use in preventing, treating, or ameliorating a cancer in a subject that has an elevated neutrophil to lymphocyte ratio.

In another embodiment, the invention relates to the use of a LAG-3 protein, or a derivative thereof that is able to bind to MHC class II molecules, in the manufacture of a medicament for the prevention, treatment, or amelioration of a cancer in a subject that has an elevated neutrophil to lymphocyte ratio.

In yet another embodiment, the invention relates to the use of a LAG-3 protein, or a derivative thereof that is able to bind to MHC class II molecules, for the prevention, treatment, or amelioration of a cancer in a subject that has an elevated neutrophil to lymphocyte ratio.

In a further embodiment, the invention provides a method of preventing, treating, or ameliorating a cancer in a subject that has an elevated neutrophil to lymphocyte ratio, the method comprising administering to the subject in need of such prevention, treatment, or amelioration a LAG-3 protein, or a derivative thereof that is able to bind to MHC class II molecules.

The neutrophil to lymphocyte ratio (NLR) is calculated as a simple ratio between the neutrophil and lymphocyte counts and is a biomarker which reflects the balance between two aspects of the immune system: acute and chronic inflammation (as indicated by the neutrophil count) and adaptive immunity (lymphocyte count). An elevated ratio reflects increased inflammation and has been associated with a poorer prognosis for patients with some types of cancer and other diseases including cardiovascular diseases, infections, and inflammatory diseases. A normal NLR in an adult, non-geriatric, patient in good health is between 0.78 and 3.53 (Forget P, Khalifa C, Defour J P, Latinne D, Van Pel M C, De Kock M. What is the normal value of the neutrophil-to-lymphocyte ratio? BMC Res Notes. 2017 Jan. 3; 10(1):12).

As defined herein, an “elevated neutrophil to lymphocyte ratio (NLR)” is greater than about 3.53 at baseline. “Baseline” means prior to commencement of treatment according to the invention.

In an embodiment, the NLR of the subject at baseline is greater than about 3.53, greater than about 3.54, greater than about 3.55, greater than about 3.56, greater than about 3.57, greater than about 3.58, greater than about 3.59, greater than about 3.60, greater than about 3.61, greater than about 3.62, greater than about 3.63, greater than about 3.64, greater than about 3.65, greater than about 3.66, greater than about 3.67, greater than about 3.68, greater than about 3.69, or greater than about 3.70.

In one embodiment, the NLR of the patient at baseline is greater than about 3.65.

Exemplary cancers that may be treated according to this embodiment of the invention include, but are not limited to, those that are described hereinabove.

In one particular embodiment, the cancer is breast cancer. Suitably, the breast cancer is hormone receptor-positive HER2-neg/low (HR⁺/HER2-neg/low) breast cancer, or triple negative breast cancer (TNBC).

Suitably, the breast cancer is hormone receptor-positive HER2-neg/low (HR⁺/HER2-neg/low) metastatic breast cancer, or metastatic triple negative breast cancer (TNBC).

Methods of Treatment in Patient Subgroup Diagnosed Less than 5 Years Ago

In one embodiment, the invention relates to a LAG-3 protein, or a derivative thereof that is able to bind to MHC class II molecules, for use in preventing, treating, or ameliorating a cancer in a subject diagnosed less than about 5 years ago.

In another embodiment, the invention relates to the use of a LAG-3 protein, or a derivative thereof that is able to bind to MHC class II molecules, in the manufacture of a medicament for the prevention, treatment, or amelioration of a cancer in a subject diagnosed less than about 5 years ago.

In yet another embodiment, the invention relates to the use of a LAG-3 protein, or a derivative thereof that is able to bind to MHC class II molecules, for the prevention, treatment, or amelioration of a cancer in a subject diagnosed less than about 5 years ago.

In a further embodiment, the invention provides a method of preventing, treating, or ameliorating a cancer in a subject diagnosed less than about 5 years ago, the method comprising administering to the subject in need of such prevention, treatment, or amelioration a LAG-3 protein, or a derivative thereof that is able to bind to MHC class II molecules.

In an embodiment, at the time of treatment, the subject was diagnosed with the cancer less than about 5 years ago, less than about 4 years ago, less than about 3 years ago, less than about 2 years ago, or less than about 1 year ago.

Exemplary cancers that may be treated according to this embodiment of the invention include, but are not limited to, those that are described hereinabove.

In one particular embodiment, the cancer is breast cancer. Suitably, the breast cancer is hormone receptor-positive HER2-neg/low (HR⁺/HER2-neg/low) breast cancer, or triple negative breast cancer (TNBC).

Suitably, the breast cancer is hormone receptor-positive HER2-neg/low (HR⁺/HER2-neg/low) metastatic breast cancer, or metastatic triple negative breast cancer (TNBC).

### Methods of Treatment in One or More Subgroups

In one embodiment, the subject has one or more of a low monocyte count, a Luminal B breast cancer, an age of less than about 85 years, has been previously treated with a CDK4/6 inhibitor, has not previously undergone treatment with a taxane chemotherapy, has an elevated neutrophil to lymphocyte ratio, and was diagnosed less than about 5 years ago.

In one particular embodiment, the subject has one or more of a low monocyte count, has not previously undergone treatment with a taxane chemotherapy, has an elevated neutrophil to lymphocyte ratio, and was diagnosed less than about 5 years ago.

In another particular embodiment, the subject has been previously treated with a CDK4/6 inhibitor and has one or more of a low monocyte count, has not previously undergone treatment with a taxane chemotherapy, has an elevated neutrophil to lymphocyte ratio, and was diagnosed less than about 5 years ago.

In one particular embodiment, the subject has not previously undergone treatment with a taxane chemotherapy and has an elevated neutrophil to lymphocyte ratio.

In another particular embodiment, the subject has been previously treated with a CDK4/6 inhibitor, and has not previously undergone treatment with a taxane chemotherapy and has an elevated neutrophil to lymphocyte ratio.

Methods of Treatment with a High Dose of LAG-3 Protein or a Derivative Thereof

In a further embodiment, the invention provides a LAG-3 protein, or a derivative thereof that is able to bind to MHC class II molecules, for use in preventing, treating, or ameliorating a cancer in a subject, wherein the LAG-3 protein, or derivative, is to be administered to the subject at a dosage of a molar equivalent of >30 mg to 200 mg of LAG-3 derivative LAG-3Ig fusion protein IMP321.

In a further embodiment, the invention provides use of a LAG-3 protein, or a derivative thereof that is able to bind to MHC class II molecules, in the manufacture of a medicament for preventing, treating, or ameliorating a cancer in a subject, wherein the LAG-3 protein, or derivative, is to be administered to the subject at a dosage of a molar equivalent of >30 mg to 200 mg of LAG-3 derivative LAG-3Ig fusion protein IMP321.

In a further embodiment, the invention provides a method of preventing, treating, or ameliorating a cancer in a subject, the method comprising administering to the subject a LAG-3 protein, or a derivative thereof that is able to bind to MHC class II molecules, at a dosage of a molar equivalent of >30 mg to 200 mg of LAG-3 derivative LAG-3Ig fusion protein IMP321.

In a further embodiment, the invention provides a LAG-3 protein, or a derivative thereof that is able to bind to MHC class II molecules, for use in preventing, treating, or ameliorating a cancer in a subject, wherein the LAG-3 protein, or derivative, is to be administered to the subject at a dosage of a molar equivalent of >30 mg to 180 mg of LAG-3 derivative LAG-3Ig fusion protein IMP321.

In a further embodiment, the invention provides use of a LAG-3 protein, or a derivative thereof that is able to bind to MHC class II molecules, in the manufacture of a medicament for preventing, treating, or ameliorating a cancer in a subject, wherein the LAG-3 protein, or derivative, is to be administered to the subject at a dosage of a molar equivalent of >30 mg to 180 mg of LAG-3 derivative LAG-3Ig fusion protein IMP321.

In a further embodiment, the invention provides a method of preventing, treating, or ameliorating a cancer in a subject, the method comprising administering to the subject a LAG-3 protein, or a derivative thereof that is able to bind to MHC class II molecules, at a dosage of a molar equivalent of >30 mg to 180 mg of LAG-3 derivative LAG-3Ig fusion protein IMP321.

In a further embodiment, the invention provides a LAG-3 protein, or a derivative thereof that is able to bind to MHC class II molecules, for use in preventing, treating, or ameliorating a cancer in a subject, wherein the LAG-3 protein, or derivative, is to be administered to the subject at a dosage of a molar equivalent of >30 mg to 150 mg of LAG-3 derivative LAG-3Ig fusion protein IMP321.

In a further embodiment, the invention provides use of a LAG-3 protein, or a derivative thereof that is able to bind to MHC class II molecules, in the manufacture of a medicament for preventing, treating, or ameliorating a cancer in a subject, wherein the LAG-3 protein, or derivative, is to be administered to the subject at a dosage of a molar equivalent of >30 mg to 150 mg of LAG-3 derivative LAG-3Ig fusion protein IMP321.

In a further embodiment, the invention provides a method of preventing, treating, or ameliorating a cancer in a subject, the method comprising administering to the subject a LAG-3 protein, or a derivative thereof that is able to bind to MHC class II molecules, at a dosage of a molar equivalent of >30 mg to 150 mg of LAG-3 derivative LAG-3Ig fusion protein IMP321.

In a further embodiment, the invention provides a LAG-3 protein, or a derivative thereof that is able to bind to MHC class II molecules, for use in preventing, treating, or ameliorating a cancer in a subject, wherein the LAG-3 protein, or derivative, is to be administered to the subject at a dosage of a molar equivalent of >30 mg to 120 mg of LAG-3 derivative LAG-3Ig fusion protein IMP321.

In a further embodiment, the invention provides use of a LAG-3 protein, or a derivative thereof that is able to bind to MHC class II molecules, in the manufacture of a medicament for preventing, treating, or ameliorating a cancer in a subject, wherein the LAG-3 protein, or derivative, is to be administered to the subject at a dosage of a molar equivalent of >30 mg to 120 mg of LAG-3 derivative LAG-3Ig fusion protein IMP321.

In a further embodiment, the invention provides a method of preventing, treating, or ameliorating a cancer in a subject, the method comprising administering to the subject a LAG-3 protein, or a derivative thereof that is able to bind to MHC class II molecules, at a dosage of a molar equivalent of >30 mg to 120 mg of LAG-3 derivative LAG-3Ig fusion protein IMP321.

In a further embodiment, the invention provides a LAG-3 protein, or a derivative thereof that is able to bind to MHC class II molecules, for use in preventing, treating, or ameliorating a cancer in a subject, wherein the LAG-3 protein, or derivative, is to be administered to the subject at a dosage of a molar equivalent of >30 mg to <120 mg of LAG-3 derivative LAG-3Ig fusion protein IMP321.

In a further embodiment, the invention provides use of a LAG-3 protein, or a derivative thereof that is able to bind to MHC class II molecules, in the manufacture of a medicament for preventing, treating, or ameliorating a cancer in a subject, wherein the LAG-3 protein, or derivative, is to be administered to the subject at a dosage of a molar equivalent of >30 mg to <120 mg of LAG-3 derivative LAG-3Ig fusion protein IMP321.

In a further embodiment, the invention provides a method of preventing, treating, or ameliorating a cancer in a subject, the method comprising administering to the subject a LAG-3 protein, or a derivative thereof that is able to bind to MHC class II molecules, at a dosage of a molar equivalent of >30 mg to <120 mg of LAG-3 derivative LAG-3Ig fusion protein IMP321.

Optionally the LAG-3 protein, or derivative thereof, is to be administered to the subject at a dosage of a molar equivalent of 40 mg to 200 mg of IMP321.

Optionally the LAG-3 protein, or derivative thereof, is to be administered to the subject at a dosage of a molar equivalent of 40 mg to 180 mg of IMP321.

Optionally the LAG-3 protein, or derivative thereof, is to be administered to the subject at a dosage of a molar equivalent of 40 mg to 150 mg of IMP321.

Optionally the LAG-3 protein, or derivative thereof, is to be administered to the subject at a dosage of a molar equivalent of 40 mg to 120 mg of IMP321.

Optionally the LAG-3 protein, or derivative thereof, is to be administered to the subject at a dosage of a molar equivalent of 50 mg to 200 mg of IMP321.

Optionally the LAG-3 protein, or derivative thereof, is to be administered to the subject at a dosage of a molar equivalent of 50 mg to 180 mg of IMP321.

Optionally the LAG-3 protein, or derivative thereof, is to be administered to the subject at a dosage of a molar equivalent of 50 mg to 150 mg of IMP321.

Optionally the LAG-3 protein, or derivative thereof, is to be administered to the subject at a dosage of a molar equivalent of 50 mg to 120 mg of IMP321.

Optionally the LAG-3 protein, or derivative thereof, is to be administered to the subject at a dosage of a molar equivalent of 50 to 100 mg of IMP321.

Optionally the LAG-3 protein, or derivative thereof, is to be administered to the subject at a dosage of a molar equivalent of 60 mg to 200 mg of IMP321.

Optionally the LAG-3 protein, or derivative thereof, is to be administered to the subject at a dosage of a molar equivalent of 60 mg to 180 mg of IMP321.

Optionally the LAG-3 protein, or derivative thereof, is to be administered to the subject at a dosage of a molar equivalent of 60 mg to 150 mg of IMP321.

Optionally the LAG-3 protein, or derivative thereof, is to be administered to the subject at a dosage of a molar equivalent of 60 mg to 120 mg of IMP321.

Optionally the LAG-3 protein, or derivative thereof, is to be administered to the subject at a dosage of a molar equivalent of 60 to 100 mg of IMP321, or 60 to 90 mg of IMP321.

Optionally the LAG-3 protein, or derivative thereof, is to be administered to the subject at a dosage of a molar equivalent of 80 to 100 mg of IMP321

Optionally the LAG-3 protein, or derivative thereof, is to be administered to the subject at a dosage of a molar equivalent of 90 mg of IMP321.

The dosage of the LAG-3 protein, or derivative thereof, may be administered to the subject in two or more separate administrations, each separate administration comprising a partial dose, to provide the full dose in combination. For example a 90 mg dose of IMP321 may be administered in two separate doses of 45 mg (for example, by s.c. injection). The separate administrations may be up to 30 minutes apart, for example, up to 15 minutes apart.

Optionally a plurality of doses of the LAG-3 protein, or derivative thereof, is to be administered to the subject.

Optionally the LAG-3 protein, or derivative thereof, is to be administered to the subject before, with, or after administration of a chemotherapy agent.

Optionally a plurality of doses of the chemotherapy agent is to be administered to the subject.

Optionally the LAG-3 protein, or derivative, is to be administered to the subject on the same day as the chemotherapy agent.

Optionally the LAG-3 protein, or derivative is to be administered to the subject on Day 1 and Day 15 of a four-week cycle, and the chemotherapy agent is to be administered to the subject on Day 1, Day 8, and Day 15 of the four-week cycle, optionally wherein the four-week cycle is repeated for four to eight cycles, preferably six cycles.

Optionally the LAG-3 protein, or derivative is to be administered to the subject on Day 1 and Day 15 of a four-week cycle, and the chemotherapy agent is to be administered to the subject on Day 1, Day 8, and Day 15 of the four-week cycle, optionally wherein the four-week cycle is repeated for 4 to 13 cycles, suitably for 6, 7, 8, 9, 10, 11, 12, or 13 cycles.

Optionally the LAG-3 protein, or derivative, is to be administered to the subject one or more times in the absence of a chemotherapy agent, after one or more dosages of the LAG-3 protein, or derivative, have been administered to the subject before, with, or after one or more dosages of the chemotherapy agent.

Optionally the LAG-3 protein, or derivative, is to be administered to the subject in the absence of a chemotherapy agent on Day 1 and Day 15 of a four-week cycle for upto nine four-week cycles.

Optionally the LAG-3 protein, or derivative, is to be administered to the subject in the absence of a chemotherapy agent on Day 1 and Day 15 of a four-week cycle for upto seven four-week cycles.

Optionally the LAG-3 protein, or derivative, is to be administered to the subject in the absence of a chemotherapy agent after doses of the LAG-3 protein, or derivative, on Day 1 and Day 15 of a four-week cycle, and doses of the chemotherapy agent on Day 1, Day 8, and Day 15 of the four-week cycle, for four to eight, preferably six, four-week cycles.

Optionally the LAG-3 protein, or derivative, is to be administered to the subject in the absence of a chemotherapy agent after doses of the LAG-3 protein, or derivative, on Day 1 and Day 15 of a four-week cycle, and doses of the chemotherapy agent on Day 1, Day 8, and Day 15 of the four-week cycle, for 4 to 13 cycles, suitably for 6, 7, 8, 9, 10, 11, 12 or 13 four-week cycles.

Optionally the chemotherapy agent is to be administered at a dosage in accordance with the approved prescribing information.

Optionally the chemotherapy agent is a taxane.

Optionally the chemotherapy agent is paclitaxel.

Optionally the LAG-3 derivative is LAG-3Ig fusion protein IMP321.

Optionally 80 mg/m² paclitaxel is to be administered to the subject intravenously on day 1, 8 and 15 in a 4-week cycle, followed by 90 mg IMP321 subcutaneously on day 1 and 15 in the 4-week cycle.

Optionally the LAG-3 derivative is LAG-3Ig fusion protein IMP321.

Optionally the cancer is a breast cancer.

Optionally the cancer is a metastatic breast cancer.

Optionally the subject is a hormone receptor-positive HER2-neg/low (HR⁺/HER2-neg/low) metastatic breast cancer patient, or a metastatic triple negative breast cancer (TNBC) patient.

Methods of Treatment with LAG-3 Protein, or a Derivative Thereof, and a Chemotherapy Agent on the Same Day

In a further embodiment, the invention provides a LAG-3 protein, or a derivative thereof that is able to bind to MHC class II molecules, for use in preventing, treating, or ameliorating a cancer in a subject, wherein the LAG-3 protein, or derivative, is to be administered to the subject on the same day as a chemotherapy agent.

In a further embodiment, the invention provides use of a LAG-3 protein, or a derivative thereof that is able to bind to MHC class II molecules, in the manufacture of a medicament for preventing, treating, or ameliorating a cancer in a subject, wherein the LAG-3 protein, or derivative, is to be administered to the subject on the same day as a chemotherapy agent.

In a further embodiment, the invention provides a method of preventing, treating, or ameliorating a cancer in a subject, the method comprising administering to the subject a LAG-3 protein, or a derivative thereof that is able to bind to MHC class II molecules, on the same day as a chemotherapy agent.

Optionally the LAG-3 protein, or derivative is to be administered to the subject on Day 1 and Day 15 of a four-week cycle, and the chemotherapy agent is to be administered to the subject on Day 1, Day 8, and Day 15 of the four-week cycle, optionally wherein the four-week cycle is repeated for four to eight cycles, preferably six cycles.

Optionally the LAG-3 protein, or derivative, is to be administered at a dosage of a molar equivalent of 6 mg to <120 mg, preferably 20 mg to 100 mg, more preferably 30 mg to 90 mg, of LAG-3 derivative LAG-3Ig fusion protein IMP321.

Optionally the chemotherapy agent is to be administered at a dosage in accordance with the approved prescribing information.

Optionally the chemotherapy agent is a taxane.

Optionally the chemotherapy agent is paclitaxel.

Optionally the LAG-3 derivative is LAG-3Ig fusion protein IMP321.

Optionally 80 mg/m² paclitaxel is to be administered to the subject intravenously on day 1, 8 and 15 in a 4-week cycle, followed by 90 mg IMP321 subcutaneously on day 1 and 15 in the 4-week cycle.

Optionally the cancer is a breast cancer.

Optionally the cancer is a metastatic breast cancer.

Optionally the subject is a hormone receptor-positive HER2-neg/low (HR⁺/HER2-neg/low) metastatic breast cancer patient, or a metastatic triple negative breast cancer (TNBC) patient.

Methods of Treatment with a LAG-3 Protein, or a Derivative Thereof, in the Absence of a Chemotherapy Agent, Following Combined Treatment with a LAG-3 Protein, or a Derivative Thereof, and a Chemotherapy Agent

In a further embodiment, the invention provides a LAG-3 protein, or a derivative thereof that is able to bind to MHC class II molecules, for use in preventing, treating, or ameliorating a cancer in a subject, wherein the LAG-3 protein, or derivative, is to be administered to the subject one or more times in the absence of a chemotherapy agent, after one or more dosages of the LAG-3 protein, or derivative, have been administered to the subject before, with, or after one or more dosages of the chemotherapy agent.

In a further embodiment, the invention provides use of a LAG-3 protein, or a derivative thereof that is able to bind to MHC class II molecules, in the manufacture of a medicament for preventing, treating, or ameliorating a cancer in a subject, wherein the LAG-3 protein, or derivative, is to be administered to the subject one or more times in the absence of a chemotherapy agent, after one or more dosages of the LAG-3 protein, or derivative, have been administered to the subject before, with, or after one or more dosages of the chemotherapy agent.

In a further embodiment, the invention provides a method of preventing, treating, or ameliorating a cancer in a subject, the method comprising administering to the subject a LAG-3 protein, or a derivative thereof that is able to bind to MHC class II molecules, one or more times in the absence of a chemotherapy agent, after one or more dosages of the LAG-3 protein, or derivative, have been administered to the subject before, with, or after one or more dosages of the chemotherapy agent.

Optionally the LAG-3 protein, or derivative, is to be administered to the subject in the absence of a chemotherapy agent on Day 1 and Day 15 of a four-week cycle for upto nine four-week cycles.

Optionally the LAG-3 protein, or derivative, is to be administered to the subject in the absence of a chemotherapy agent on Day 1 and Day 15 of a four-week cycle for upto seven four-week cycles.

Optionally the LAG-3 protein, or derivative, is to be administered to the subject in the absence of a chemotherapy agent after doses of the LAG-3 protein, or derivative, on Day 1 and Day 15 of a four-week cycle, and doses of the chemotherapy agent on Day 1, Day 8, and Day 15 of the four-week cycle, for four to eight, preferably six, four-week cycles.

Optionally the LAG-3 protein, or derivative, is to be administered to the subject in the absence of a chemotherapy agent after doses of the LAG-3 protein, or derivative, on Day 1 and Day 15 of a four-week cycle, and doses of the chemotherapy agent on Day 1, Day 8, and Day 15 of the four-week cycle, for 4 to 13, suitably 6, 7, 8, 9, 10, 11, 12 or 13 four-week cycles.

Optionally the LAG-3 protein, or derivative, is to be administered at a dosage of a molar equivalent of 1 mg to 200 mg, 6 mg to 200 mg, 6 mg to <120 mg, 20 mg to 100 mg, 30 mg to 90 mg, about 30 mg, or about 90 mg of LAG-3 derivative LAG-3Ig fusion protein IMP321.

Optionally the chemotherapy agent is to be administered at a dosage in accordance with the approved prescribing information.

Optionally the chemotherapy agent is a taxane.

Optionally the chemotherapy agent is paclitaxel.

Optionally the LAG-3 derivative is LAG-3Ig fusion protein IMP321.

Optionally 80 mg/m² paclitaxel is to be administered to the subject intravenously on day 1, 8 and 15 in a 4-week cycle, followed by 90 mg IMP321 subcutaneously on day 1 and 15 in the 4-week cycle.

Optionally the cancer is a breast cancer.

Optionally the cancer is a metastatic breast cancer.

Optionally the subject is a hormone receptor-positive HER2-neg/low (HR⁺/HER2-neg/low) metastatic breast cancer patient, or a metastatic triple negative breast cancer (TNBC) patient.

### Methods of Treatment of a Patient Sub-Group

In a further embodiment, the invention provides a LAG-3 protein, or a derivative thereof that is able to bind to MHC class II molecules, for use in preventing, treating, or ameliorating a metastatic breast cancer in a subject, wherein the subject is a hormone receptor-positive HER2-neg/low (HR⁺/HER2-neg/low) metastatic breast cancer patient, or a metastatic triple negative breast cancer (TNBC) patient.

In a further embodiment, the invention provides use of a LAG-3 protein, or a derivative thereof that is able to bind to MHC class II molecules, in the manufacture of a medicament for preventing, treating, or ameliorating a metastatic breast cancer in a subject, wherein the subject is a hormone receptor-positive HER2-neg/low (HR⁺/HER2-neg/low) metastatic breast cancer patient, or a metastatic triple negative breast cancer (TNBC) patient.

In a further embodiment, the invention provides a method of preventing, treating, or ameliorating a metastatic breast cancer in a subject, the method comprising administering to the subject a LAG-3 protein, or a derivative thereof that is able to bind to MHC class II molecules, wherein the subject is a hormone receptor-positive HER2-neg/low (HR⁺/HER2-neg/low) metastatic breast cancer patient, or a metastatic triple negative breast cancer (TNBC) patient.

### LAG-3 Protein and Derivatives

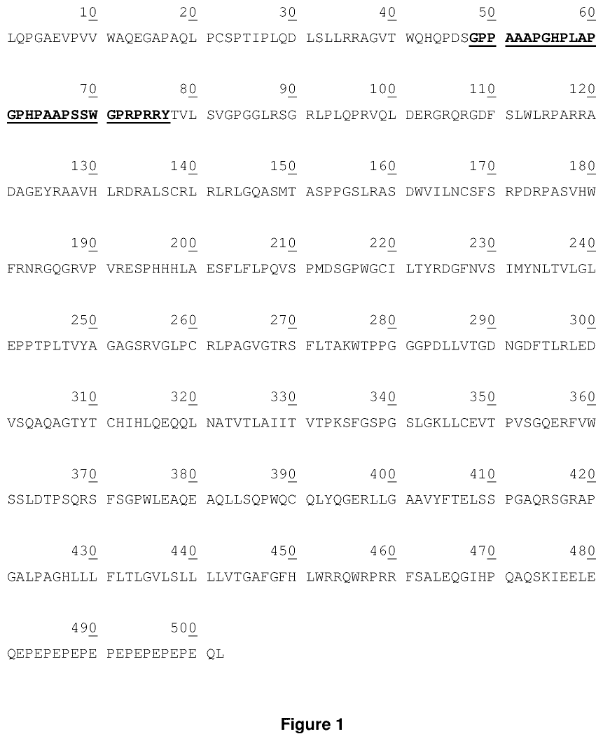

According to embodiments of the invention, the LAG-3 protein may be an isolated natural or recombinant LAG-3 protein. The LAG-3 protein may comprise an amino acid sequence of LAG-3 protein from any suitable species, such as a primate or murine LAG-3 protein, but preferably a human LAG-3 protein. The amino acid sequence of human and murine LAG-3 protein is provided in FIG. 1 of Huard et al (Proc. Natl. Acad. Sci. USA, 11:5744-5749, 1997). The sequence of human LAG-3 protein is repeated in FIG. 1 herein (SEQ ID NO: 1). The amino acid sequences of the four extracellular Ig superfamily domains (D1, D2, D3, and D4) of human LAG-3 are also identified in FIG. 1 of Huard et al., at amino acid residues: 1-149 (D1); 150-239 (D2); 240-330 (D3); and 331-412 (D4).

Derivatives of LAG-3 protein include soluble fragments, variants, or mutants of LAG-3 protein that are able to bind to MHC class II molecules. Several derivatives of LAG-3 protein are known that are able to bind to MHC class II molecules. Many examples of such derivatives are described in Huard et al (Proc. Natl. Acad. Sci. USA, 11:5744-5749, 1997). This document describes characterization of the MHC class II binding site on LAG-3 protein. Methods for making mutants of LAG-3 are described, as well as a quantitative cellular adhesion assay for determining the ability of LAG-3 mutants to bind to class II-positive Daudi cells. Binding of several different mutants of LAG-3 to MHC class II molecules was determined. Some mutations were able to reduce class II binding, while other mutations increased the affinity of LAG-3 for class II molecules. Many of the residues essential for binding of LAG-3 to MHC class II proteins are clustered at the base of a large 30 amino acid extra-loop structure in the LAG-3 D1 domain. The amino acid sequence of the extra-loop structure of the D1 domain of human LAG-3 protein is GPPAAAPGHPLAPGPHPAAPSSWGPRPRRY (SEQ ID NO:2). The amino acid sequence of the extra-loop structure of the D1 domain of human LAG-3 protein is shown underlined in bold in FIG. 1.

In an embodiment of the invention, the derivative of LAG-3 protein comprises the 30 amino acid extra-loop sequence of the human LAG-3 D1 domain, or a variant of such sequence with one or more amino acid substitutions. The variant may comprise an amino acid sequence that has at least 70%, 80%, 90%, or 95% amino acid identity with the 30 amino acid extra-loop sequence of the human LAG-3 D1 domain. Optionally the amino acid substitution is a conservative amino acid substitution.

The derivative of LAG-3 protein may comprise an amino acid sequence of domain D1 and optionally D2, or domains D1 and D2, of LAG-3 protein, preferably human LAG-3 protein.

The derivative of LAG-3 protein may comprise an amino acid sequence that has at least 70%, 80%, 90%, or 95% amino acid identity with domain D1 and optionally D2, or domains D1 and D2, of LAG-3 protein, preferably human LAG-3 protein.

The derivative of LAG-3 protein may comprise an amino acid sequence of domains D1, D2, and D3, domains D1, D2, D3 and optionally D4, or domains D1, D2, D3 and D4, of LAG-3 protein, preferably human LAG-3 protein.

The derivative of LAG-3 protein may comprise an amino acid sequence that has at least 70%, 80%, 90%, or 95% amino acid identity with domains D1, D2 and D3, domains D1, D2, D3 and optionally D4, or with domains D1, D2, D3 and D4, of LAG-3 protein, preferably human LAG-3.

Sequence identity between amino acid sequences can be determined by comparing an alignment of the sequences. When an equivalent position in the compared sequences is occupied by the same amino acid, then the molecules are identical at that position. Scoring an alignment as a percentage of identity is a function of the number of identical amino acids at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include MatGat (Campanella et al., 2003, BMC Bioinformatics 4:29; program available from http://bitincka.com/ledion/matgat), Gap (Needleman & Wunsch, 1970, J. Mol. Biol. 48:443-453), FASTA (Altschul et al., 1990, J. Mol. Biol. 215:403-410; program available from http://www.ebi.ac.uk/fasta), Clustal W 2.0 and X 2.0 (Larkin et al., 2007, Bioinformatics 23:2947-2948; program available from http://www.ebi.ac.uk/tools/clustalw2) and EMBOSS Pairwise Alignment Algorithms (Needleman & Wunsch, 1970, supra; Kruskal, 1983, In: Time warps, string edits and macromolecules: the theory and practice of sequence comparison, Sankoff & Kruskal (eds), pp 1-44, Addison Wesley; programs available from http://www.ebi.ac.uk/tools/emboss/align). All programs may be run using default parameters.

For example, sequence comparisons may be undertaken using the “needle” method of the EMBOSS Pairwise Alignment Algorithms, which determines an optimum alignment (including gaps) of two sequences when considered over their entire length and provides a percentage identity score. Default parameters for amino acid sequence comparisons (“Protein Molecule” option) may be Gap Extend penalty: 0.5, Gap Open penalty: 10.0, Matrix: Blosum 62.

The sequence comparison may be performed over the full length of the reference sequence.

The derivative of LAG-3 protein may be fused to Immunoglobulin Fc amino acid sequence, preferably human IgG1 Fc amino acid sequence, optionally by a linker amino acid sequence.

The ability of a derivative of LAG-3 protein to bind to MHC class II molecules may be determined using a quantitative cellular adhesion assay as described in Huard et al (Proc. Natl. Acad. Sci. USA, 11:5744-5749, 1997). The affinity of a derivative of LAG-3 protein for MHC class II molecules may be at least 20%, 30%, 40%, 50%, 60%, 70%, 80%, 90%, or 100% of the affinity of human LAG-3 protein for MHC class II molecules.

Preferably, the affinity of a derivative of LAG-3 protein for MHC class II molecules is at least 50%, 60%, 70%, 80%, 90%, 95%, 99%, or 100% of the affinity of human LAG-3 protein for MHC class II molecules.

Examples of suitable derivatives of LAG-3 protein that are able to bind to MHC class II molecules include derivatives comprising:
amino acid residues 23 to 448 of the human LAG-3 sequence;
amino acid sequence of domains D1 and D2 of LAG-3;
amino acid sequence of domains D1 and D2 of LAG-3 with an amino acid substitution at one or more of the following positions: position 30 where ASP is substituted with ALA; position 56 where HIS is substituted with ALA; position 73 where ARG is substituted with GLU; position 75 where ARG is substituted with ALA or GLU; position 76 where ARG is substituted with GLU; or position 103 where ARG is substituted with ALA; and
a recombinant soluble human LAG-3Ig fusion protein (IMP321)—a 160-kDa dimer produced in Chinese hamster ovary cells transfected with a plasmid encoding for the extracellular domain of hLAG-3 fused to the human IgG1 Fc. The sequence of IMP321 is given in SEQ ID NO: 17 of US 2011/0008331.

In an embodiment, the subject is a mammal, preferably a human.

According to the invention, the LAG-3 protein or derivative thereof is administered in a therapeutically effective amount. A “therapeutically effective amount” refers to an amount of the active ingredient sufficient to have a therapeutic effect upon administration. Effective amounts of the active ingredient will vary, for example, with the particular disease or diseases being treated, the severity of the disease, the duration of the treatment, and characteristics of the patient (e.g. sex, age, height and weight).

In an embodiment, the LAG-3 protein or derivative thereof is administered at a dose which is a molar equivalent of about 0.1 mg to about 200 mg, about 1 mg to about 200 mg, about 6 mg to about 200 mg, about 10 mg to about 200 mg, about 20 mg to about 200 mg, about 30 mg to about 200 mg, >30 mg to about 200 mg, about 40 mg to about 200 mg, about 50 mg to about 200 mg, or about 80 mg to about 200 mg, of the LAG-3 derivative LAG-3Ig fusion protein IMP321.

In an embodiment, the LAG-3 protein or derivative thereof is administered at a dose which is a molar equivalent of about 0.1 mg to about 180 mg, about 1 mg to about 180 mg, about 6 mg to about 180 mg, about 10 mg to about 180 mg, about 20 mg to about 180 mg, about 30 mg to about 180 mg, >30 mg to about 180 mg, about 40 mg to about 180 mg, about 50 mg to about 180 mg, or about 80 mg to about 180 mg, of the LAG-3 derivative LAG-3Ig fusion protein IMP321.

In an embodiment, the LAG-3 protein or derivative thereof is administered at a dose which is a molar equivalent of about 0.1 mg to about 120 mg, about 1 mg to about 120 mg, about 6 mg to about 120 mg, about 10 mg to about 120 mg, about 20 mg to about 120 mg, about 30 mg to about 120 mg, >30 mg to about 120 mg, about 40 mg to about 120 mg, about 50 mg to about 120 mg, or about 80 mg to about 120 mg, of the LAG-3 derivative LAG-3Ig fusion protein IMP321.

In an embodiment, the LAG-3 protein or derivative thereof is administered at a dose which is a molar equivalent of about 0.1 mg to about 60 mg, about 6 mg to about 60 mg, about 10 mg to about 50 mg, about 20 mg to about 40 mg, about 25 mg to about 35 mg, or about 30 mg of the LAG-3 derivative LAG-3Ig fusion protein IMP321.

In another embodiment, the LAG-3 protein or derivative thereof is administered at a dose which is a molar equivalent of about 25 mg, about 26 mg, about 27 mg, about 28 mg, about 29 mg, about 30 mg, about 31 mg, about 32 mg, about 33 mg, about 34 mg, or about 35 mg of the LAG-3 derivative LAG-3Ig fusion protein IMP321.

Suitably, the LAG-3 protein or derivative thereof is administered at a dose which is a molar equivalent of about 30 mg of the LAG-3 derivative LAG-3Ig fusion protein IMP321.

In yet another embodiment, the LAG-3 protein or derivative thereof is administered at a dose which is a molar equivalent from about 25 mg to about 60 mg, such as about 25 mg, about 30 mg, about 35 mg, about 40 mg, about 45 mg, about 50 mg, about 55 mg, or about 60 mg, of the LAG-3 derivative LAG-3Ig fusion protein IMP321.

In one embodiment, the LAG-3 protein or derivative thereof is IMP321 and is administered at a dose of about 0.1 mg to about 60 mg, about 6 mg to about 60 mg, about 10 mg to about 50 mg, about 20 mg to about 40 mg, about 25 mg to about 35 mg, or about 30 mg.

In another embodiment, the IMP321 is administered at a dose of about 25 mg, about 26 mg, about 27 mg, about 28 mg, about 29 mg, about 30 mg, about 31 mg, about 32 mg, about 33 mg, about 34 mg, or about 35 mg.

Suitably, IMP321 is administered at a dose of about 30 mg.

In other embodiments, IMP321 is administered at a dose from about 25 mg to about 60 mg, such as about 25 mg, about 30 mg, about 35 mg, about 40 mg, about 45 mg, about 50 mg, about 55 mg, or about 60 mg.

Doses of 6-30 mg per subcutaneous (s.c.) injection of IMP321 have been shown, thus far, to be safe and provide an acceptable systemic exposure based on the results of pharmacokinetics data obtained in metastatic renal cell cancer patients. A blood concentration of IMP321 superior to 1 ng/ml for at least 24 hours after s.c. injection is obtained in patients injected with IMP321 doses of more than 6 mg. No dose limiting toxicity has been observed to date.

In an embodiment, the LAG-3 protein or derivative thereof is administered about once every week to the subject. In another embodiment, the LAG-3 protein or derivative thereof is administered about once every two weeks to the subject. In yet another embodiment, the LAG-3 protein or derivative thereof is administered about once every three weeks to the subject. In a further embodiment, the LAG-3 protein or derivative thereof is administered about once every four weeks to the subject. In yet a further embodiment, the LAG-3 protein or derivative thereof is administered about once every month to a subject. As will be appreciated by those of skill in the art, the precise treatment regimen will vary and be adapted according to the particular cancer being treated and characteristics of the patient.

In one embodiment, the LAG-3 protein or derivative thereof is present as the sole active ingredient. In another embodiment, the LAG-3 protein or derivative thereof is present in the absence of any additional antigen added to the pharmaceutical composition or medicament.

Combination Treatment with Chemotherapy in One or More Subgroups

In an embodiment, the LAG-3 protein or derivative thereof is administered in combination with a chemotherapy agent.

Suitable chemotherapy agents include, but are not limited to, alkylating agents, plant alkaloids, antitumor antibiotics, antimetabolites, topoisomerase inhibitors, and miscellaneous antineoplastics.

Suitably, the chemotherapy agent is an alkylating agent. Exemplary alkylating agents include mustard gas derivatives such mechlorethamine, cyclophosphamide, chlorambucil, melphalan, and ifosfamide; ethylenimines such as thiotepa and hexamethylmelamine; alkylsulfonates such as busulfan; hydrazines and triazines such as altretamine, procarbazine, dacarbazine and temozolomide; nitrosureas such as carmustine, lomustine and streptozocin; and metal salts such as carboplatin, cisplatin, and oxaliplatin.

Suitably, the chemotherapy agent is a plant alkaloid. Exemplary plant alkaloids include vinca alkaloids such as vincristine, vinblastine and vinorelbine; taxanes such as paclitaxel, nab-paclitaxel, docetaxel, cabazitaxel, larotaxel, milataxel, ortataxel, taxoprexin, opaxio, tesetaxel, and BMS-184476; podophyllotoxins such as etoposide and tenisopide; and camptothecan analogs such as irinotecan and topotecan.

Suitably, the chemotherapy agent is an antitumor antibiotic. Exemplary antitumor antibiotics include anthracyclines such as doxorubicin, daunorubicin, epirubicin, mitoxantrone, and idarubicin; chromomycins such as dactinomycin and plicamycin; and miscellaneous antitumor antibiotics such as mitomycin and bleomycin.

Suitably, the chemotherapy agent is an antimetabolite. Exemplary antimetabolites include folic acid antagonists such as methotrexate; pyrimidine antagonists such as 5-fluorouracil, foxuridine, cytarabine, capecitabine and gemcitabine; purine antagonists such as 6-mercaptopurine and 6-thioguanine; and adenosine deaminase inhibitors such as cladribine, fludarabine, nelarabine and pentostatin.

Suitably, the chemotherapy agent is a topoisomerase inhibitor. Exemplary topoisomerase inhibitors include topoisomerase I inhibitors such as irinotecan and topotecan; and topoisomerase II inhibitors such as amsacrine, etoposide, etoposide phosphate and teniposide.

Suitably, the chemotherapy agent is a miscellaneous antineoplastic.

Exemplary miscellaneous antineoplastics include ribonucleotide reductase inhibitors such as hydroxyurea; adrenocortical steroid inhibitors such as mitotane; enzymes such as asparaginase and pegaspargase; antimicrotubule agents such as estramustine; and retinoids such bexarotene, isotretinoin and tretinoin.

In one particular embodiment, the chemotherapy agent is a taxane. In an embodiment, the taxane is paclitaxel, nab-paclitaxel, docetaxel, cabazitaxel, larotaxel, milataxel, ortataxel, taxoprexin, opaxio, tesetaxel, or BMS-184476.

In another embodiment, the taxane is paclitaxel.

The chemotherapy agent is administered in a therapeutically effective amount. A therapeutically effective amount refers to an amount of the chemotherapy agent sufficient to have a therapeutic effect upon administration. Effective amounts of the chemotherapy agent will vary with the chemotherapy agent selected, the particular disease or diseases being treated, the severity of the disease, the duration of the treatment, and characteristics of the patient (e.g. sex, age, height and weight). Suitably, the chemotherapy agent is administered in accordance with the approved prescribing information for the chemotherapy agent.

In some embodiments, the chemotherapy agent is administered parenterally (including by subcutaneous, intravenous, or intramuscular injection) or orally. Suitably, the chemotherapy is administered intravenously.

In an embodiment, the LAG-3 protein or derivative thereof is administered before, with or after administration of the chemotherapy agent. In another embodiment, the LAG-3 protein or derivative thereof is administered after administration of the chemotherapy agent.

In one embodiment, the LAG-3 protein or derivative thereof and the chemotherapy agent are packaged separately. That is, in this embodiment, the LAG-3 protein or derivative thereof and the chemotherapy agent are separate unit dosage forms, which would typically (but not necessarily) be sourced from different suppliers, and then used in the methods of the invention.

In another embodiment, the LAG-3 protein or derivative thereof and the chemotherapy agent are in the form of a combined preparation.

The components of the “combined preparation” may be present: (i) in one combined unit dosage form known as a fixed dose combination (FDC), or (ii) as a first unit dosage form of component (a) and a separate, second unit dosage form of component (b) packaged together known as a kit-of-parts. The ratio of the total amounts of the combination component (a) to the combination component (b) to be administered in the combined preparation can be varied, for example, in order to cope with the needs of a patient sub-population to be treated, or the needs of the patient, which can be due, for example, to the particular disease, age, sex, or body weight of the patient.

That is, the combined preparation according to the invention may take the form of a pharmaceutical composition comprising the LAG-3 protein or derivative thereof and the chemotherapy agent or, alternatively, as a kit-of-parts comprising the LAG-3 protein or derivative thereof and the chemotherapy agent as separate components, but packaged together.

The kit-of-parts may comprise a plurality of doses of the LAG-3 protein or derivative thereof and/or a plurality of doses of the chemotherapy agent.

In one embodiment, the invention relates to a LAG-3 protein, or a derivative thereof that is able to bind to MHC class II molecules, and a chemotherapy agent, for use in preventing, treating, or ameliorating a cancer in a subject with a low monocyte count.

In another embodiment, the invention relates to the use of a LAG-3 protein, or a derivative thereof that is able to bind to MHC class II molecules, and a chemotherapy agent, in the manufacture of a medicament for the prevention, treatment, or amelioration of a cancer in a subject with a low monocyte count.

In yet another embodiment, the invention relates to the use of a LAG-3 protein, or a derivative thereof that is able to bind to MHC class II molecules, and a chemotherapy agent, for the prevention, treatment, or amelioration of a cancer in a subject with a low monocyte count.

In a further embodiment, the invention provides a method of preventing, treating, or ameliorating a cancer in a subject with a low monocyte count, the method comprising administering to the subject in need of such prevention, treatment, or amelioration a LAG-3 protein, or a derivative thereof that is able to bind to MHC class II molecules, and a chemotherapy agent.

In yet a further embodiment, the invention relates to a LAG-3 protein, or a derivative thereof that is able to bind to MHC class II molecules, for use in preventing, treating, or ameliorating a cancer in a subject with a low monocyte count, wherein the LAG-3 protein, or a derivative thereof is to be administered before, with or after administration of a chemotherapy agent.

In another embodiment, the invention relates to the use of a LAG-3 protein, or a derivative thereof that is able to bind to MHC class II molecules, in the manufacture of a medicament for the prevention, treatment, or amelioration of a cancer in a subject with a low monocyte count, wherein the LAG-3 protein or derivative thereof is to be administered before, with or after administration of a chemotherapy agent.

In an embodiment, the invention provides a method of preventing, treating, or ameliorating a cancer in a subject with a low monocyte count, the method comprising administering to the subject in need of such prevention, treatment, or amelioration a LAG-3 protein, or a derivative thereof that is able to bind to MHC class II molecules, and a chemotherapy agent, wherein the LAG-3 protein or derivative thereof is administered before, with or after administration of the chemotherapy agent.

In one embodiment, the invention relates to a LAG-3 protein, or a derivative thereof that is able to bind to MHC class II molecules, and a chemotherapy agent, for use in preventing, treating, or ameliorating a Luminal B breast cancer in a subject.

In another embodiment, the invention relates to the use of a LAG-3 protein, or a derivative thereof that is able to bind to MHC class II molecules, and a chemotherapy agent, in the manufacture of a medicament for the prevention, treatment, or amelioration of a Luminal B breast cancer in a subject.

In yet another embodiment, the invention relates to the use of a LAG-3 protein, or a derivative thereof that is able to bind to MHC class II molecules, and a chemotherapy agent, for the prevention, treatment, or amelioration of a Luminal B breast cancer in a subject.

In a further embodiment, the invention provides a method of preventing, treating, or ameliorating a Luminal B breast cancer in a subject, the method comprising administering to the subject in need of such prevention, treatment, or amelioration a LAG-3 protein, or a derivative thereof that is able to bind to MHC class II molecules, and a chemotherapy agent.

In yet a further embodiment, the invention relates to a LAG-3 protein, or a derivative thereof that is able to bind to MHC class II molecules, for use in preventing, treating, or ameliorating a Luminal B breast cancer in a subject, wherein the LAG-3 protein or derivative thereof is to be administered before, with or after administration of a chemotherapy agent.

In another embodiment, the invention relates to the use of a LAG-3 protein, or a derivative thereof that is able to bind to MHC class II molecules, in the manufacture of a medicament for the prevention, treatment, or amelioration of a Luminal B breast cancer in a subject, wherein the LAG-3 protein or derivative thereof is to be administered before, with or after administration of a chemotherapy agent.

In an embodiment, the invention provides a method of preventing, treating, or ameliorating a Luminal B breast cancer in a subject, the method comprising administering to the subject in need of such prevention, treatment, or amelioration a LAG-3 protein, or a derivative thereof that is able to bind to MHC class II molecules, and a chemotherapy agent, wherein the LAG-3 protein or derivative thereof is administered before, with or after administration of the chemotherapy agent.

In one embodiment, the invention relates to a LAG-3 protein, or a derivative thereof that is able to bind to MHC class II molecules, and a chemotherapy agent, for use in preventing, treating, or ameliorating a cancer in a subject with an age of less than about 85 years.

In another embodiment, the invention relates to the use of a LAG-3 protein, or a derivative thereof that is able to bind to MHC class II molecules, and a chemotherapy agent, in the manufacture of a medicament for the prevention, treatment, or amelioration of a cancer in a subject with an age of less than about 85 years.

In yet another embodiment, the invention relates to the use of a LAG-3 protein, or a derivative thereof that is able to bind to MHC class II molecules, and a chemotherapy agent, for the prevention, treatment, or amelioration of a cancer in a subject with an age of less than about 85 years.

In a further embodiment, the invention provides a method of preventing, treating, or ameliorating a cancer in a subject with an age of less than about 85 years, the method comprising administering to the subject in need of such prevention, treatment, or amelioration a LAG-3 protein, or a derivative thereof that is able to bind to MHC class II molecules, and a chemotherapy agent.

In yet a further embodiment, the invention relates to a LAG-3 protein, or a derivative thereof that is able to bind to MHC class II molecules, for use in preventing, treating, or ameliorating a cancer in a subject with an age of less than about 85 years, wherein the LAG-3 protein or derivative thereof is to be administered before, with or after administration of a chemotherapy agent.

In another embodiment, the invention relates to the use of a LAG-3 protein, or a derivative thereof that is able to bind to MHC class II molecules, in the manufacture of a medicament for the prevention, treatment, or amelioration of a cancer in a subject with an age of less than about 85 years, wherein the LAG-3 protein or derivative thereof is to be administered before, with or after administration of a chemotherapy agent.

In an embodiment, the invention provides a method of preventing, treating, or ameliorating a cancer in a subject with an age of less than about 85 years, the method comprising administering to the subject in need of such prevention, treatment, or amelioration a LAG-3 protein, or a derivative thereof that is able to bind to MHC class II molecules, and a chemotherapy agent, wherein the LAG-3 protein or derivative thereof is administered before, with or after administration of the chemotherapy agent.

In one embodiment, the invention relates to a LAG-3 protein, or a derivative thereof that is able to bind to MHC class II molecules, and a chemotherapy agent, for use in preventing, treating, or ameliorating a cancer in a subject previously treated with a CDK4/6 inhibitor.

In another embodiment, the invention relates to the use of a LAG-3 protein, or a derivative thereof that is able to bind to MHC class II molecules, and a chemotherapy agent, in the manufacture of a medicament for the prevention, treatment, or amelioration of a cancer in a subject previously treated with a CDK4/6 inhibitor.

In yet another embodiment, the invention relates to the use of a LAG-3 protein, or a derivative thereof that is able to bind to MHC class II molecules, and a chemotherapy agent, for the prevention, treatment, or amelioration of a cancer in a subject previously treated with a CDK4/6 inhibitor.

In a further embodiment, the invention provides a method of preventing, treating, or ameliorating a cancer in a subject previously treated with a CDK4/6 inhibitor, the method comprising administering to the subject in need of such prevention, treatment, or amelioration a LAG-3 protein, or a derivative thereof that is able to bind to MHC class II molecules, and a chemotherapy agent.

In yet a further embodiment, the invention relates to a LAG-3 protein, or a derivative thereof that is able to bind to MHC class II molecules, for use in preventing, treating, or ameliorating a cancer in a subject previously treated with a CDK4/6 inhibitor, wherein the LAG-3 protein or derivative thereof is to be administered before, with or after administration of a chemotherapy agent.

In another embodiment, the invention relates to the use of a LAG-3 protein, or a derivative thereof that is able to bind to MHC class II molecules, in the manufacture of a medicament for the prevention, treatment, or amelioration of a cancer in a subject previously treated with a CDK4/6 inhibitor, wherein the LAG-3 protein or derivative thereof is to be administered before, with or after administration of a chemotherapy agent.

In an embodiment, the invention provides a method of preventing, treating, or ameliorating a cancer in a subject previously treated with a CDK4/6 inhibitor, the method comprising administering to the subject in need of such prevention, treatment, or amelioration a LAG-3 protein, or a derivative thereof that is able to bind to MHC class II molecules, and a chemotherapy agent, wherein the LAG-3 protein or derivative thereof is administered before, with or after administration of the chemotherapy agent.

In one embodiment, the invention relates to a LAG-3 protein, or a derivative thereof that is able to bind to MHC class II molecules, and a chemotherapy agent, for use in preventing, treating, or ameliorating a cancer in a subject that has not previously undergone treatment with a taxane chemotherapy.

In another embodiment, the invention relates to the use of a LAG-3 protein, or a derivative thereof that is able to bind to MHC class II molecules, and a chemotherapy agent, in the manufacture of a medicament for the prevention, treatment, or amelioration of a cancer in a subject that has not previously undergone treatment with a taxane chemotherapy.

In yet another embodiment, the invention relates to the use of a LAG-3 protein, or a derivative thereof that is able to bind to MHC class II molecules, and a chemotherapy agent, for the prevention, treatment, or amelioration of a cancer in a subject that has not previously undergone treatment with a taxane chemotherapy.

In a further embodiment, the invention provides a method of preventing, treating, or ameliorating a cancer in a subject that has not previously undergone treatment with a taxane chemotherapy, the method comprising administering to the subject in need of such prevention, treatment, or amelioration a LAG-3 protein, or a derivative thereof that is able to bind to MHC class II molecules, and a chemotherapy agent.

In yet a further embodiment, the invention relates to a LAG-3 protein, or a derivative thereof that is able to bind to MHC class II molecules, for use in preventing, treating, or ameliorating a cancer in a subject that has not previously undergone treatment with a taxane chemotherapy, wherein the LAG-3 protein or derivative thereof is to be administered before, with or after administration of a chemotherapy agent.

In another embodiment, the invention relates to the use of a LAG-3 protein, or a derivative thereof that is able to bind to MHC class II molecules, in the manufacture of a medicament for the prevention, treatment, or amelioration of a cancer in a subject that has not previously undergone treatment with a taxane chemotherapy, wherein the LAG-3 protein or derivative thereof is to be administered before, with or after administration of a chemotherapy agent.

In an embodiment, the invention provides a method of preventing, treating, or ameliorating a cancer in a subject that has not previously undergone treatment with a taxane chemotherapy, the method comprising administering to the subject in need of such prevention, treatment, or amelioration a LAG-3 protein, or a derivative thereof that is able to bind to MHC class II molecules, and a chemotherapy agent, wherein the LAG-3 protein or derivative thereof is administered before, with or after administration of the chemotherapy agent.

In one embodiment, the invention relates to a LAG-3 protein, or a derivative thereof that is able to bind to MHC class II molecules, and a chemotherapy agent, for use in preventing, treating, or ameliorating a cancer in a subject that has an elevated neutrophil to lymphocyte ratio.

In another embodiment, the invention relates to the use of a LAG-3 protein, or a derivative thereof that is able to bind to MHC class II molecules, and a chemotherapy agent, in the manufacture of a medicament for the prevention, treatment, or amelioration of a cancer in a subject that has an elevated neutrophil to lymphocyte ratio.

In yet another embodiment, the invention relates to the use of a LAG-3 protein, or a derivative thereof that is able to bind to MHC class II molecules, and a chemotherapy agent, for the prevention, treatment, or amelioration of a cancer in a subject that has an elevated neutrophil to lymphocyte ratio.

In a further embodiment, the invention provides a method of preventing, treating, or ameliorating a cancer in a subject that has an elevated neutrophil to lymphocyte ratio, the method comprising administering to the subject in need of such prevention, treatment, or amelioration a LAG-3 protein, or a derivative thereof that is able to bind to MHC class II molecules, and a chemotherapy agent.

In yet a further embodiment, the invention relates to a LAG-3 protein, or a derivative thereof that is able to bind to MHC class II molecules, for use in preventing, treating, or ameliorating a cancer in a subject that has an elevated neutrophil to lymphocyte ratio, wherein the LAG-3 protein or derivative thereof is to be administered before, with or after administration of a chemotherapy agent.

In another embodiment, the invention relates to the use of a LAG-3 protein, or a derivative thereof that is able to bind to MHC class II molecules, in the manufacture of a medicament for the prevention, treatment, or amelioration of a cancer in a subject that has an elevated neutrophil to lymphocyte ratio, wherein the LAG-3 protein or derivative thereof is to be administered before, with or after administration of a chemotherapy agent.

In an embodiment, the invention provides a method of preventing, treating, or ameliorating a cancer in a subject that has an elevated neutrophil to lymphocyte ratio, the method comprising administering to the subject in need of such prevention, treatment, or amelioration a LAG-3 protein, or a derivative thereof that is able to bind to MHC class II molecules, and a chemotherapy agent, wherein the LAG-3 protein or derivative thereof is administered before, with or after administration of the chemotherapy agent.

In one embodiment, the invention relates to a LAG-3 protein, or a derivative thereof that is able to bind to MHC class II molecules, and a chemotherapy agent, for use in preventing, treating, or ameliorating a cancer in a subject that was diagnosed less than about 5 years ago.

In another embodiment, the invention relates to the use of a LAG-3 protein, or a derivative thereof that is able to bind to MHC class II molecules, and a chemotherapy agent, in the manufacture of a medicament for the prevention, treatment, or amelioration of a cancer in a subject that was diagnosed less than about 5 years ago.

In yet another embodiment, the invention relates to the use of a LAG-3 protein, or a derivative thereof that is able to bind to MHC class II molecules, and a chemotherapy agent, for the prevention, treatment, or amelioration of a cancer in a subject that was diagnosed less than about 5 years ago.

In a further embodiment, the invention provides a method of preventing, treating, or ameliorating a cancer in a subject that was diagnosed less than about 5 years ago, the method comprising administering to the subject in need of such prevention, treatment, or amelioration a LAG-3 protein, or a derivative thereof that is able to bind to MHC class II molecules, and a chemotherapy agent.

In yet a further embodiment, the invention relates to a LAG-3 protein, or a derivative thereof that is able to bind to MHC class II molecules, for use in preventing, treating, or ameliorating a cancer in a subject that was diagnosed less than about 5 years ago, wherein the LAG-3 protein or derivative thereof is to be administered before, with or after administration of a chemotherapy agent.

In another embodiment, the invention relates to the use of a LAG-3 protein, or a derivative thereof that is able to bind to MHC class II molecules, in the manufacture of a medicament for the prevention, treatment, or amelioration of a cancer in a subject that was diagnosed less than about 5 years ago, wherein the LAG-3 protein or derivative thereof is to be administered before, with or after administration of a chemotherapy agent.

In an embodiment, the invention provides a method of preventing, treating, or ameliorating a cancer in a subject that was diagnosed less than about 5 years ago, the method comprising administering to the subject in need of such prevention, treatment, or amelioration a LAG-3 protein, or a derivative thereof that is able to bind to MHC class II molecules, and a chemotherapy agent, wherein the LAG-3 protein or derivative thereof is administered before, with or after administration of the chemotherapy agent.

In one embodiment, the invention relates to a LAG-3 protein, or a derivative thereof that is able to bind to MHC class II molecules, and a chemotherapy agent, for use in preventing, treating, or ameliorating a cancer in a subject, wherein the subject has one or more of a low monocyte count, a Luminal B breast cancer, an age of less than about 85 years, has been previously treated with a CDK4/6 inhibitor, has not previously undergone treatment with a taxane chemotherapy, has an elevated neutrophil to lymphocyte ratio, and was diagnosed less than about 5 years ago.

In another embodiment, the invention relates to the use of a LAG-3 protein, or a derivative thereof that is able to bind to MHC class II molecules, and a chemotherapy agent, in the manufacture of a medicament for the prevention, treatment, or amelioration of a cancer in a subject, wherein the subject has one or more of a low monocyte count, a Luminal B breast cancer, an age of less than about 85 years, has been previously treated with a CDK4/6 inhibitor, has not previously undergone treatment with a taxane chemotherapy, has an elevated neutrophil to lymphocyte ratio, and was diagnosed less than about 5 years ago.

In yet another embodiment, the invention relates to the use of a LAG-3 protein, or a derivative thereof that is able to bind to MHC class II molecules, and a chemotherapy agent, for the prevention, treatment, or amelioration of a cancer in a subject, wherein the subject has one or more of a low monocyte count, a Luminal B breast cancer, an age of less than about 85 years, has been previously treated with a CDK4/6 inhibitor, has not previously undergone treatment with a taxane chemotherapy, has an elevated neutrophil to lymphocyte ratio, and was diagnosed less than about 5 years ago.

In a further embodiment, the invention provides a method of preventing, treating, or ameliorating a cancer in a subject, the method comprising administering to the subject in need of such prevention, treatment, or amelioration a LAG-3 protein, or a derivative thereof that is able to bind to MHC class II molecules, and a chemotherapy agent, wherein the subject has one or more of a low monocyte count, a Luminal B breast cancer, an age of less than about 85 years, has been previously treated with a CDK4/6 inhibitor, has not previously undergone treatment with a taxane chemotherapy, has an elevated neutrophil to lymphocyte ratio, and was diagnosed less than about 5 years ago.

In yet a further embodiment, the invention relates to a LAG-3 protein, or a derivative thereof that is able to bind to MHC class II molecules, for use in preventing, treating, or ameliorating a cancer in a subject, wherein the LAG-3 protein or derivative thereof is to be administered before, with or after administration of a chemotherapy agent, and wherein the subject has one or more of a low monocyte count, a Luminal B breast cancer, an age of less than about 85 years, has been previously treated with a CDK4/6 inhibitor, has not previously undergone treatment with a taxane chemotherapy, has an elevated neutrophil to lymphocyte ratio, and was diagnosed less than about 5 years ago.

In another embodiment, the invention relates to the use of a LAG-3 protein, or a derivative thereof that is able to bind to MHC class II molecules, in the manufacture of a medicament for the prevention, treatment, or amelioration of a cancer in a subject, wherein the LAG-3 protein or derivative thereof is to be administered before, with or after administration of a chemotherapy agent, and wherein the subject has one or more of a low monocyte count, a Luminal B breast cancer, an age of less than about 85 years, has been previously treated with a CDK4/6 inhibitor, has not previously undergone treatment with a taxane chemotherapy, has an elevated neutrophil to lymphocyte ratio, and was diagnosed less than about 5 years ago.

In an embodiment, the invention provides a method of preventing, treating, or ameliorating a cancer in a subject, the method comprising administering to the subject in need of such prevention, treatment, or amelioration a LAG-3 protein, or a derivative thereof that is able to bind to MHC class II molecules, and a chemotherapy agent, wherein the LAG-3 protein or derivative thereof is administered before, with or after administration of the chemotherapy agent, and wherein the subject has one or more of a low monocyte count, a Luminal B breast cancer, an age of less than about 85 years, has been previously treated with a CDK4/6 inhibitor, has not previously undergone treatment with a taxane chemotherapy, has an elevated neutrophil to lymphocyte ratio, and was diagnosed less than about 5 years ago.

In one particular embodiment, the subject has one or more of a low monocyte count, has not previously undergone treatment with a taxane chemotherapy, has an elevated neutrophil to lymphocyte ratio, and was diagnosed less than about 5 years ago.

In another particular embodiment, the subject has been previously treated with a CDK4/6 inhibitor and has one or more of a low monocyte count, has not previously undergone treatment with a taxane chemotherapy, has an elevated neutrophil to lymphocyte ratio, and was diagnosed less than about 5 years ago.

In one particular embodiment, the subject has not previously undergone treatment with a taxane chemotherapy and has an elevated neutrophil to lymphocyte ratio.

In another particular embodiment, the subject has been previously treated with a CDK4/6 inhibitor, and has not previously undergone treatment with a taxane chemotherapy and has an elevated neutrophil to lymphocyte ratio.

In a further embodiment, the invention relates to a LAG-3 protein, or a derivative thereof that is able to bind to MHC class II molecules, and a chemotherapy agent, for use in preventing, treating, or ameliorating a cancer in a subject, wherein the LAG-3 protein, or derivative, is to be administered to the subject at a dosage of a molar equivalent of >30 mg to <200 mg of LAG-3 derivative LAG-3Ig fusion protein IMP321.

In a further embodiment, the invention relates to a LAG-3 protein, or a derivative thereof that is able to bind to MHC class II molecules, and a chemotherapy agent, for use in preventing, treating, or ameliorating a cancer in a subject, wherein the LAG-3 protein, or derivative, is to be administered to the subject at a dosage of a molar equivalent of >30 mg to <120 mg of LAG-3 derivative LAG-3Ig fusion protein IMP321.

In another embodiment, the invention relates to the use of a LAG-3 protein, or a derivative thereof that is able to bind to MHC class II molecules, and a chemotherapy agent, in the manufacture of a medicament for the prevention, treatment, or amelioration of a cancer in a subject, wherein the LAG-3 protein, or derivative, is to be administered to the subject at a dosage of a molar equivalent of >30 mg to <200 mg of LAG-3 derivative LAG-3Ig fusion protein IMP321.

In another embodiment, the invention relates to the use of a LAG-3 protein, or a derivative thereof that is able to bind to MHC class II molecules, and a chemotherapy agent, in the manufacture of a medicament for the prevention, treatment, or amelioration of a cancer in a subject, wherein the LAG-3 protein, or derivative, is to be administered to the subject at a dosage of a molar equivalent of >30 mg to <120 mg of LAG-3 derivative LAG-3Ig fusion protein IMP321.

In a further embodiment, the invention provides a method of preventing, treating, or ameliorating a cancer in a subject, the method comprising administering to the subject in need of such prevention, treatment, or amelioration a LAG-3 protein, or a derivative thereof that is able to bind to MHC class II molecules, and a chemotherapy agent, wherein the LAG-3 protein, or derivative, is administered to the subject at a dosage of a molar equivalent of >30 mg to <200 mg of LAG-3 derivative LAG-3Ig fusion protein IMP321.

In a further embodiment, the invention provides a method of preventing, treating, or ameliorating a cancer in a subject, the method comprising administering to the subject in need of such prevention, treatment, or amelioration a LAG-3 protein, or a derivative thereof that is able to bind to MHC class II molecules, and a chemotherapy agent, wherein the LAG-3 protein, or derivative, is administered to the subject at a dosage of a molar equivalent of >30 mg to <120 mg of LAG-3 derivative LAG-3Ig fusion protein IMP321.

In yet a further embodiment, the invention relates to a LAG-3 protein, or a derivative thereof that is able to bind to MHC class II molecules, for use in preventing, treating, or ameliorating a cancer in a subject, wherein the LAG-3 protein or derivative thereof is to be administered before, with or after administration of a chemotherapy agent, and wherein the LAG-3 protein, or derivative, is to be administered to the subject at a dosage of a molar equivalent of >30 mg to <200 mg of LAG-3 derivative LAG-3Ig fusion protein IMP321.

In yet a further embodiment, the invention relates to a LAG-3 protein, or a derivative thereof that is able to bind to MHC class II molecules, for use in preventing, treating, or ameliorating a cancer in a subject, wherein the LAG-3 protein or derivative thereof is to be administered before, with or after administration of a chemotherapy agent, and wherein the LAG-3 protein, or derivative, is to be administered to the subject at a dosage of a molar equivalent of >30 mg to <120 mg of LAG-3 derivative LAG-3Ig fusion protein IMP321.

In another embodiment, the invention relates to the use of a LAG-3 protein, or a derivative thereof that is able to bind to MHC class II molecules, in the manufacture of a medicament for the prevention, treatment, or amelioration of a cancer in a subject, wherein the LAG-3 protein or derivative thereof is to be administered before, with or after administration of a chemotherapy agent, and wherein the LAG-3 protein, or derivative, is to be administered to the subject at a dosage of a molar equivalent of >30 mg to <200 mg of LAG-3 derivative LAG-3Ig fusion protein IMP321.

In another embodiment, the invention relates to the use of a LAG-3 protein, or a derivative thereof that is able to bind to MHC class II molecules, in the manufacture of a medicament for the prevention, treatment, or amelioration of a cancer in a subject, wherein the LAG-3 protein or derivative thereof is to be administered before, with or after administration of a chemotherapy agent, and wherein the LAG-3 protein, or derivative, is to be administered to the subject at a dosage of a molar equivalent of >30 mg to <120 mg of LAG-3 derivative LAG-3Ig fusion protein IMP321.

In a further embodiment, the invention provides a method of preventing, treating, or ameliorating a cancer in a subject who has already been administered a chemotherapy agent, the method comprising administering to the subject in need of such prevention, treatment, or amelioration a LAG-3 protein, or a derivative thereof that is able to bind to MHC class II molecules, wherein the LAG-3 protein, or derivative, is administered to the subject at a dosage of a molar equivalent of >30 mg to <200 mg of LAG-3 derivative LAG-3Ig fusion protein IMP321.

In a further embodiment, the invention provides a method of preventing, treating, or ameliorating a cancer in a subject who has already been administered a chemotherapy agent, the method comprising administering to the subject in need of such prevention, treatment, or amelioration a LAG-3 protein, or a derivative thereof that is able to bind to MHC class II molecules, wherein the LAG-3 protein, or derivative, is administered to the subject at a dosage of a molar equivalent of >30 mg to <120 mg of LAG-3 derivative LAG-3Ig fusion protein IMP321.

In yet a further embodiment, the invention relates to a chemotherapy agent for use in preventing, treating, or ameliorating a cancer in a subject, wherein the chemotherapy agent is to be administered before, with or after administration of a LAG-3 protein, or a derivative thereof that is able to bind to MHC class II molecules, at a dosage of a molar equivalent of >30 mg to <200 mg of LAG-3 derivative LAG-3Ig fusion protein IMP321.

In yet a further embodiment, the invention relates to a chemotherapy agent for use in preventing, treating, or ameliorating a cancer in a subject, wherein the chemotherapy agent is to be administered before, with or after administration of a LAG-3 protein, or a derivative thereof that is able to bind to MHC class II molecules, at a dosage of a molar equivalent of >30 mg to <120 mg of LAG-3 derivative LAG-3Ig fusion protein IMP321.

In another embodiment, the invention relates to the use of a chemotherapy agent in the manufacture of a medicament for the prevention, treatment, or amelioration of a cancer in a subject, wherein the chemotherapy agent is to be administered before, with or after administration of a LAG-3 protein, or a derivative thereof that is able to bind to MHC class II molecules, at a dosage of a molar equivalent of >30 mg to <200 mg of LAG-3 derivative LAG-3Ig fusion protein IMP321.

In another embodiment, the invention relates to the use of a chemotherapy agent in the manufacture of a medicament for the prevention, treatment, or amelioration of a cancer in a subject, wherein the chemotherapy agent is to be administered before, with or after administration of a LAG-3 protein, or a derivative thereof that is able to bind to MHC class II molecules, at a dosage of a molar equivalent of >30 mg to <120 mg of LAG-3 derivative LAG-3Ig fusion protein IMP321.

In a further embodiment, the invention provides a method of preventing, treating, or ameliorating a cancer in a subject who has already been administered a LAG-3 protein, or a derivative thereof that is able to bind to MHC class II molecules, at a dosage of a molar equivalent of >30 mg to <200 mg of LAG-3 derivative LAG-3Ig fusion protein IMP321, the method comprising administering to the subject in need of such prevention, treatment, or amelioration a chemotherapy agent.

In a further embodiment, the invention provides a method of preventing, treating, or ameliorating a cancer in a subject who has already been administered a LAG-3 protein, or a derivative thereof that is able to bind to MHC class II molecules, at a dosage of a molar equivalent of >30 mg to <120 mg of LAG-3 derivative LAG-3Ig fusion protein IMP321, the method comprising administering to the subject in need of such prevention, treatment, or amelioration a chemotherapy agent.

Optionally the LAG-3 protein, or derivative thereof, is to be administered to the subject at a dosage of a molar equivalent of 40 mg to 200 mg of IMP321.

Optionally the LAG-3 protein, or derivative thereof, is to be administered to the subject at a dosage of a molar equivalent of 40 mg to 180 mg of IMP321.

Optionally the LAG-3 protein, or derivative thereof, is to be administered to the subject at a dosage of a molar equivalent of 40 mg to 150 mg of IMP321.

Optionally the LAG-3 protein, or derivative thereof, is to be administered to the subject at a dosage of a molar equivalent of 40 mg to 120 mg of IMP321.

Optionally the LAG-3 protein, or derivative thereof, is to be administered to the subject at a dosage of a molar equivalent of 50 mg to 200 mg of IMP321.

Optionally the LAG-3 protein, or derivative thereof, is to be administered to the subject at a dosage of a molar equivalent of 50 mg to 180 mg of IMP321.

Optionally the LAG-3 protein, or derivative thereof, is to be administered to the subject at a dosage of a molar equivalent of 50 mg to 150 mg of IMP321.

Optionally the LAG-3 protein, or derivative thereof, is to be administered to the subject at a dosage of a molar equivalent of 50 mg to 120 mg of IMP321.

Optionally the LAG-3 protein, or derivative thereof, is to be administered to the subject at a dosage of a molar equivalent of 50 to 100 mg of IMP321.

Optionally the LAG-3 protein, or derivative thereof, is to be administered to the subject at a dosage of a molar equivalent of 60 to 100 mg of IMP321, or 60 to 90 mg of IMP321.

Optionally the LAG-3 protein, or derivative thereof, is to be administered to the subject at a dosage of a molar equivalent of 80 to 100 mg of IMP321

Optionally the LAG-3 protein, or derivative thereof, is to be administered to the subject at a dosage of a molar equivalent of 90 mg of IMP321.

The dosage of the LAG-3 protein, or derivative thereof, may be administered to the subject in two or more separate administrations, each separate administration comprising a partial dose, to provide the full dose in combination. For example a 90 mg dose of IMP321 may be administered in two separate doses of 45 mg (for example, by s.c. injection). The separate administrations may be up to 30 minutes apart, for example, up to 15 minutes apart.

Optionally a plurality of doses of the LAG-3 protein, or derivative thereof, is to be administered to the subject.

Optionally a plurality of doses of the chemotherapy agent is to be administered to the subject.

Optionally the LAG-3 protein, or derivative, is to be administered to the subject on the same day as the chemotherapy agent.

Optionally the LAG-3 protein, or derivative is to be administered to the subject on Day 1 and Day 15 of a four-week cycle, and the chemotherapy agent is to be administered to the subject on Day 1, Day 8, and Day 15 of the four-week cycle, optionally wherein the four-week cycle is repeated for four to eight cycles, preferably six cycles.

Optionally the LAG-3 protein, or derivative is to be administered to the subject on Day 1 and Day 15 of a four-week cycle, and the chemotherapy agent is to be administered to the subject on Day 1, Day 8, and Day 15 of the four-week cycle, optionally wherein the four-week cycle is repeated for 4 to 13 cycles, suitably for 6, 7, 8, 9, 10, 11, 12 or 13 cycles.

Optionally the LAG-3 protein, or derivative, is to be administered to the subject one or more times in the absence of a chemotherapy agent, after one or more dosages of the LAG-3 protein, or derivative, have been administered to the subject before, with, or after one or more dosages of the chemotherapy agent.

Optionally the LAG-3 protein, or derivative, is to be administered to the subject in the absence of a chemotherapy agent on Day 1 and Day 15 of a four-week cycle for upto nine four-week cycles.

Optionally the LAG-3 protein, or derivative, is to be administered to the subject in the absence of a chemotherapy agent on Day 1 and Day 15 of a four-week cycle for upto seven four-week cycles.

Optionally the LAG-3 protein, or derivative, is to be administered to the subject in the absence of a chemotherapy agent after doses of the LAG-3 protein, or derivative, on Day 1 and Day 15 of a four-week cycle, and doses of the chemotherapy agent on Day 1, Day 8, and Day 15 of the four-week cycle, for four to eight, preferably six, four-week cycles.

Optionally the LAG-3 protein, or derivative, is to be administered to the subject in the absence of a chemotherapy agent after doses of the LAG-3 protein, or derivative, on Day 1 and Day 15 of a four-week cycle, and doses of the chemotherapy agent on Day 1, Day 8, and Day 15 of the four-week cycle, for 4 to 13, suitably 6, 7, 8, 9, 10, 11, 12 or 13, four-week cycles.

Optionally the chemotherapy agent is to be administered at a dosage in accordance with the approved prescribing information.

Optionally the chemotherapy agent is a taxane.

Optionally the chemotherapy agent is paclitaxel.

Optionally the LAG-3 derivative is LAG-3Ig fusion protein IMP321.

Optionally 80 mg/m² paclitaxel is to be administered to the subject intravenously on day 1, 8 and 15 in a 4-week cycle, followed by 90 mg IMP321 subcutaneously on day 1 and 15 in the 4-week cycle.

Optionally the LAG-3 derivative is LAG-3Ig fusion protein IMP321.

Optionally the cancer is a breast cancer.

Optionally the cancer is a metastatic breast cancer.

Optionally the subject is a hormone receptor-positive HER2-neg/low (HR⁺/HER2-neg/low) metastatic breast cancer patient, or a metastatic triple negative breast cancer (TNBC) patient.

In a further embodiment, the invention relates to a LAG-3 protein, or a derivative thereof that is able to bind to MHC class II molecules, and a chemotherapy agent, for use in preventing, treating, or ameliorating a cancer in a subject, wherein the LAG-3 protein, or derivative, is to be administered to the subject on the same day as the chemotherapy agent.

In another embodiment, the invention relates to the use of a LAG-3 protein, or a derivative thereof that is able to bind to MHC class II molecules, and a chemotherapy agent, in the manufacture of a medicament for the prevention, treatment, or amelioration of a cancer in a subject, wherein the LAG-3 protein, or derivative, is to be administered to the subject on the same day as the chemotherapy agent.

In a further embodiment, the invention provides a method of preventing, treating, or ameliorating a cancer in a subject, the method comprising administering to the subject in need of such prevention, treatment, or amelioration a LAG-3 protein, or a derivative thereof that is able to bind to MHC class II molecules, and a chemotherapy agent, wherein the LAG-3 protein, or derivative, is administered to the subject on the same day as the chemotherapy agent.

In yet a further embodiment, the invention relates to a LAG-3 protein, or a derivative thereof that is able to bind to MHC class II molecules, for use in preventing, treating, or ameliorating a cancer in a subject, wherein the LAG-3 protein or derivative thereof is to be administered before, with or after administration of a chemotherapy agent, and wherein the LAG-3 protein, or derivative, is to be administered to the subject on the same day as the chemotherapy agent.

In another embodiment, the invention relates to the use of a LAG-3 protein, or a derivative thereof that is able to bind to MHC class II molecules, in the manufacture of a medicament for the prevention, treatment, or amelioration of a cancer in a subject, wherein the LAG-3 protein or derivative thereof is to be administered before, with or after administration of a chemotherapy agent, and wherein the LAG-3 protein, or derivative, is to be administered to the subject on the same day as the chemotherapy agent.

In a further embodiment, the invention provides a method of preventing, treating, or ameliorating a cancer in a subject who has already been administered a chemotherapy agent, the method comprising administering to the subject in need of such prevention, treatment, or amelioration a LAG-3 protein, or a derivative thereof that is able to bind to MHC class II molecules, wherein the LAG-3 protein, or derivative, is administered to the subject on the same day as the chemotherapy agent.

In yet a further embodiment, the invention relates to a chemotherapy agent for use in preventing, treating, or ameliorating a cancer in a subject, wherein the chemotherapy agent is to be administered before, with or after administration of a LAG-3 protein, or a derivative thereof that is able to bind to MHC class II molecules, and wherein the chemotherapy agent is to be administered to the subject on the same day as the LAG-3 protein, or derivative.

In another embodiment, the invention relates to the use of a chemotherapy agent in the manufacture of a medicament for the prevention, treatment, or amelioration of a cancer in a subject, wherein the chemotherapy agent is to be administered before, with or after administration of a LAG-3 protein, or a derivative thereof that is able to bind to MHC class II molecules, and wherein the chemotherapy agent is to be administered to the subject on the same day as the LAG-3 protein, or derivative.

In a further embodiment, the invention provides a method of preventing, treating, or ameliorating a cancer in a subject who has already been administered a LAG-3 protein, or a derivative thereof that is able to bind to MHC class II molecules, the method comprising administering to the subject in need of such prevention, treatment, or amelioration a chemotherapy agent, wherein the chemotherapy agent is to be administered to the subject on the same day as the LAG-3 protein, or derivative.

Optionally the LAG-3 protein, or derivative is to be administered to the subject on Day 1 and Day 15 of a four-week cycle, and the chemotherapy agent is to be administered to the subject on Day 1, Day 8, and Day 15 of the four-week cycle, optionally wherein the four-week cycle is repeated for four to eight cycles, preferably six cycles.

Optionally the LAG-3 protein, or derivative is to be administered to the subject on Day 1 and Day 15 of a four-week cycle, and the chemotherapy agent is to be administered to the subject on Day 1, Day 8, and Day 15 of the four-week cycle, optionally wherein the four-week cycle is repeated for 4 to 13 cycles, suitably for 6, 7, 8, 9, 10, 11, 12 or 13 cycles.

Optionally the LAG-3 protein, or derivative, is to be administered at a dosage of a molar equivalent of 1 mg to 200 mg, 6 mg to 200 mg, 6 mg to <120 mg, 20 mg to 100 mg, 30 mg to 90 mg, about 30 mg, or about 90 mg of LAG-3 derivative LAG-3Ig fusion protein IMP321.

Optionally the chemotherapy agent is to be administered at a dosage in accordance with the approved prescribing information.

Optionally the chemotherapy agent is a taxane.

Optionally the chemotherapy agent is paclitaxel.

Optionally the LAG-3 derivative is LAG-3Ig fusion protein IMP321.

Optionally 80 mg/m² paclitaxel is to be administered to the subject intravenously on day 1, 8 and 15 in a 4-week cycle, followed by 90 mg IMP321 subcutaneously on day 1 and 15 in the 4-week cycle.

Optionally the cancer is a breast cancer.

Optionally the cancer is a metastatic breast cancer.

Optionally the subject is a hormone receptor-positive HER2-neg/low (HR⁺/HER2-neg/low) metastatic breast cancer patient, or a metastatic triple negative breast cancer (TNBC) patient.

### Dosing Regimen of Combination Treatment

In one embodiment, the LAG-3 protein or derivative thereof is administered to the subject after administration of the chemotherapy agent and within about 12 to about 96 hours, about 12 to about 48 hours, or about 24 hours, of administration of the chemotherapy agent.

According to another embodiment of the invention, combination treatment with chemo-immunotherapy comprises 6 cycles of 4 weeks. Patients receive weekly paclitaxel at Days 1, 8 and 15 with adjunctive treatment with the LAG-3 protein or derivative thereof on Days 2 and 16 of each 4-week cycle. After completion of the 6-cycle chemo-immunotherapy phase, responding or stable patients receive LAG-3 protein or derivative thereof every 4 weeks during a maintenance phase for a period of up to 12 injections (48 weeks).

In another embodiment, the chemo-immunotherapy combination treatment comprises 7 cycles of 4 weeks, or 8 cycles of 4 weeks, or 9 cycles of 4 weeks, or 10 cycles of 4 weeks, or 11 cycles of 4 weeks, or 12 cycles of 4 weeks (extended combination treatment).

In other embodiments, after the chemo-immunotherapy phase, responding or stable patients receive LAG-3 protein or derivative thereof about every week, or about every 2 weeks, or about every 3 weeks, during a maintenance phase for up to about 48 weeks, or up to 96 weeks.

In another embodiment, the invention relates to the use of a LAG-3 protein or derivative thereof as a maintenance therapy following cancer treatment according to embodiments of the invention. Suitably, during maintenance therapy, the LAG-3 protein or derivative thereof is administered about every 1 week, or about every 2 weeks, or about every 3 weeks, or about every 4 weeks, up to about 48 weeks, or up to 96 weeks.

In a further embodiment, the invention relates to a LAG-3 protein, or a derivative thereof that is able to bind to MHC class II molecules, for use in preventing, treating, or ameliorating a cancer in a subject, wherein the LAG-3 protein, or derivative, is to be administered to the subject on the same day as a chemotherapy agent.

Optionally the LAG-3 protein, or derivative is to be administered to the subject on Day 1 and Day 15 of a four-week cycle, and the chemotherapy agent is to be administered to the subject on Day 1, Day 8, and Day 15 of the four-week cycle, optionally wherein the four-week cycle is repeated for four to eight cycles, preferably six cycles.

Optionally the LAG-3 protein, or derivative is to be administered to the subject on Day 1 and Day 15 of a four-week cycle, and the chemotherapy agent is to be administered to the subject on Day 1, Day 8, and Day 15 of the four-week cycle, optionally wherein the four-week cycle is repeated for 4, 5, 6, 7, 8, 9, 10, 11, 12 or 13 cycles, suitably for 6, 7, 8, 9, 10, 11, 12 or 13 cycles.

Optionally the LAG-3 protein, or derivative is to be administered to the subject on Day 1 and Day 15 of a four-week cycle, and the chemotherapy agent is to be administered to the subject on Day 1, Day 8, and Day 15 of the four-week cycle, optionally wherein the four-week cycle is repeated for up to 13 cycles (52 weeks).

The length of treatment with the combination of the LAG-3 protein, or derivative and the chemotherapy agent will depend on the extent to which the chemotherapy agent is well tolerated by the patient. Preferably at least 6 four-week cycles (24 weeks) of treatment with the LAG-3 protein, or derivative, and the chemotherapy agent are administered.

Optionally 80 mg/m² paclitaxel is to be administered to the subject intravenously on day 1, 8 and 15 in a 4-week cycle, followed by 90 mg IMP321 subcutaneously on day 1 and 15 in the 4-week cycle.

In a further embodiment, the invention provides a LAG-3 protein, or a derivative thereof that is able to bind to MHC class II molecules, for use in preventing, treating, or ameliorating a cancer in a subject, wherein the LAG-3 protein, or derivative, is to be administered to the subject one or more times in the absence of a chemotherapy agent, after one or more dosages of the LAG-3 protein, or derivative, have been administered to the subject before, with, or after one or more dosages of the chemotherapy agent.

Optionally the LAG-3 protein, or derivative, is to be administered to the subject in the absence of a chemotherapy agent on Day 1 and Day 15 of a four-week cycle for upto nine four-week cycles.

Optionally the LAG-3 protein, or derivative, is to be administered to the subject in the absence of a chemotherapy agent on Day 1 and Day 15 of a four-week cycle for upto seven four-week cycles.

Optionally the LAG-3 protein, or derivative, is to be administered to the subject in the absence of a chemotherapy agent after doses of the LAG-3 protein, or derivative, on Day 1 and Day 15 of a four-week cycle, and doses of the chemotherapy agent on Day 1, Day 8, and Day 15 of the four-week cycle, for four to eight, preferably six, four-week cycles.

Optionally the LAG-3 protein, or derivative, is to be administered to the subject in the absence of a chemotherapy agent after doses of the LAG-3 protein, or derivative, on Day 1 and Day 15 of a four-week cycle, and doses of the chemotherapy agent on Day 1, Day 8, and Day 15 of the four-week cycle, for 4 to 13, suitably 6, 7, 8, 9, 10, 11, 12 or 13, four-week cycles.

Optionally, the combination of the chemo-immunotherapy phase (chemo-IO) and the maintenance phase (IO) of treatment is for up to 13 four-week cycles (52 weeks) in total.

Optionally, the subject has not previously undergone treatment with chemotherapy for metastatic disease.

Optionally, the hormone receptor-positive HER2-neg/low (HR⁺/HER2-neg/low) metastatic breast cancer patient is endocrine resistant.

Optionally, the hormone receptor-positive HER2-neg/low (HR⁺/HER2-neg/low) metastatic breast cancer patient has been previously treated with a CDK4/6 inhibitor.

Optionally, the hormone receptor-positive HER2-neg/low (HR⁺/HER2-neg/low) metastatic breast cancer patient is endocrine resistant and has been previously treated with a CDK4/6 inhibitor.

Optionally, the hormone receptor-positive HER2-neg/low (HR⁺/HER2-neg/low) metastatic breast cancer patient is endocrine resistant, has been previously treated with a CDK4/6 inhibitor, and is from 18 to 65 years of age.

Optionally, the metastatic triple negative breast cancer (TNBC) patient is ineligible for anti-PD-1 or anti-PD-L1 based therapy (e.g. a combination of anti-PD-1/PD-L1 therapy and chemotherapy).

Optionally, the metastatic triple negative breast cancer (TNBC) patient is PD-L1 positive or PD-L1 negative.

Optionally, the metastatic triple negative breast cancer (TNBC) patient is PD-L1 negative.

Optionally, the metastatic triple negative breast cancer (TNBC) patient is from 18 to 65 years of age.

In one particular embodiment, the invention relates to a method of preventing, treating, or ameliorating a metastatic breast cancer in a subject, the method comprising administering to the subject in need of such prevention, treatment, or amelioration a LAG-3 protein, or a derivative thereof that is able to bind to MHC class II molecules, and a chemotherapy agent,
wherein the subject is a hormone receptor-positive HER2-neg/low (HR+/HER2-neg/low) metastatic breast cancer patient or a metastatic triple negative breast cancer (TNBC) patient,
wherein the LAG-3 protein, or derivative thereof, is administered to the subject on the same day as the chemotherapy agent,
wherein the LAG-3 protein, or derivative thereof, is administered to the subject in a dose which is a molar equivalent of about 6 mg to about 200 mg of the LAG-3 derivative LAG-Ig fusion protein IM321, and
wherein the LAG-3 protein, or derivative thereof, and the chemotherapy agent are administered to the subject for at least six 4-week cycles (24 weeks) of treatment.

### Further Patient Subgroups

In other embodiments, one or more further patient subgroups are selected for treatment. Such subgroups include, for example, patients who have a higher or lower initial performance status, patients who have previously received extensive exposure to corticosteroids, and patients with a low BMI e.g. <30 kg/m².

### Pharmaceutical Compositions

The LAG-3 protein or derivative thereof and, where applicable, the chemotherapy agent are formulated with a pharmaceutically acceptable carrier, excipient, or diluent to provide a pharmaceutical composition. Typically these will be formulated as separate pharmaceutical compositions, although in the case of a fixed dose combination, the LAG-3 protein or derivative thereof and the chemotherapy agent will be formulated together, along with a pharmaceutically acceptable carrier, excipient, or diluent. The separate pharmaceutical compositions may be packaged together in the form of a kit-of-parts.

In general, the LAG-3 protein or derivative thereof and, where applicable, the chemotherapy agent may be administered by known means, in any suitable pharmaceutical composition, by any suitable route.

Suitable pharmaceutical compositions may be prepared using conventional methods known to those in the field of pharmaceutical formulation and described in the relevant texts and literature, for example, in Remington: The Science and Practice of Pharmacy (Easton, Pa.: Mack Publishing Co., 1995).

It is especially advantageous to formulate compositions of the invention in a unit dosage form for ease of administration and uniformity of dosage. The term “unit dosage form” as used herein refers to physically discrete units suited as unitary dosages for the individuals to be treated. That is, the compositions are formulated into discrete dosage units each containing a predetermined “unit dosage” quantity of an active agent calculated to produce the desired therapeutic effect in association with the required pharmaceutical carrier, excipient or diluent. The specifications of unit dosage forms of the invention are dependent on the unique characteristics of the active agent to be delivered. Dosages can further be determined by reference to the usual dose and manner of administration of the ingredients. It should be noted that, in some cases, two or more individual dosage units in combination provide a therapeutically effective amount of the active agent.

Preparations according to the invention for parenteral administration include sterile aqueous and non-aqueous solutions, suspensions, and emulsions. Injectable aqueous solutions contain the active agent in water-soluble form. Examples of non-aqueous solvents or vehicles include fatty oils, such as olive oil and corn oil, synthetic fatty acid esters, such as ethyl oleate or triglycerides, low molecular weight alcohols such as propylene glycol, synthetic hydrophilic polymers such as polyethylene glycol, liposomes, and the like. Parenteral formulations may also contain adjuvants such as solubilizers, preservatives, wetting agents, emulsifiers, dispersants, and stabilizers, and aqueous suspensions may contain substances that increase the viscosity of the suspension, such as sodium carboxymethyl cellulose, sorbitol, and dextran. Injectable formulations may be rendered sterile by incorporation of a sterilizing agent, filtration through a bacteria-retaining filter, irradiation, or heat. They can also be manufactured using a sterile injectable medium. The active agent may also be in dried, e.g., lyophilized, form that may be rehydrated with a suitable vehicle immediately prior to administration via injection.

### EXAMPLES

Embodiments of the invention are now described, by way of example only, with reference to the accompanying drawings in which:

### Example 1—Active Immunotherapy Paclitaxel (AIPAC) in HER2⁻/HR⁺ Metastatic Breast Cancer (MBC)

A multicentre, placebo-controlled, double-blind, 1:1 randomised Phase IIb study in female HER2 negative hormone receptor-positive metastatic breast cancer patients was conducted. The study comprised two stages:
stage 1 which was an open-label, safety run-in stage consisting of cohorts 1 and 2 to confirm the recommended Phase II dose (RPTD) of IMP321 in combination with paclitaxel; and
stage 2 which was a placebo-controlled, double-blind randomisation stage, paclitaxel+IMP321 at the RPTD compared to paclitaxel+placebo.

### Experimental

### IMP321 Group: Paclitaxel+IMP321 at the RPTD of 30 mg (114 Patients):

The chemo-immunotherapy phase consisted of 6 cycles of 4 weeks. Patients received weekly paclitaxel at Days 1, 8 and 15 with adjunctive treatment of study agent (IMP321) on Days 2 and 16 of each 4-week cycle. After completion of the 6-cycle chemo-immunotherapy phase, responding or stable patients received study agent (IMP321) every 4 weeks during the maintenance phase for an additional period of up to 12 injections.

### Placebo Group: Paclitaxel+Placebo (112 Patients):

The chemo-immunotherapy phase consisted of 6 cycles of 4 weeks. Patients received weekly paclitaxel at Days 1, 8 and 15 with adjunctive treatment of study agent (placebo) on Days 2 and 16 of each 4-week cycle.

After completion of the 6-cycle chemo-immunotherapy phase, responding or stable patients received study agent (placebo) every 4 weeks during the maintenance phase for an additional period of up to 12 injections.

### Results

226 patients were included in the full analysis set [IMP321 (n=114); placebo (n=112)]. Patients were endocrine resistant (84%), pre-treated with CDK4/6 inhibitors (44.2%) and post-study treatment was similar.

TABLE 1
Efficacy measures from univariate analysis in pre-defined sub-
groups (defined prior to unblinding) from treatment with IMP321

| | | p-value from | Hazard |
|---|---|---|---|
| | Absolute gain in | univariate | Ratio |
| Sub-group | median OS | analysis | [95% CI] |
| Overall population | +2.9 months | 0.197 | 0.88 |
| <65 years | +7.5 months | 0.017 | 0.66 |
| Low baseline | +19.6 months | 0.008 | 0.44 |
| monocytes | | | |
| Luminal B | +4.2 months | 0.049 | 0.67 |
| No prior taxane | +4.8 months | 0.076 | 0.74 |
| therapy | | | |

Database cut-off date was May 14, 2021 (73% of events) with minimum follow up of 22 months

Exploratory univariate analysis (analysis groups defined prior to unblinding) showed that young patients (<65 years), those with low baseline monocytes (<0.25/nL), or breast cancer subtype Luminal B had a significant and clinically meaningful improvement in median overall survival (OS) from treatment with IMP321. In addition, multivariate analysis was done using backward selection (p>0.15) from the univariate cox model.

TABLE 2
Efficacy measures from multivariate analysis in sub-
groups (with p < 0.15) from treatment with IMP321

| | | p-value from | Hazard |
|---|---|---|---|
| | Absolute gain in | univariate | Ratio |
| Sub-group | median OS | analysis | [95% CI] |
| High (>3.65) NLR | +6.9 months | 0.012 | 0.61 |
| at baseline | | | |
| No prior taxane | +4.8 months | 0.076 | 0.74 |
| therapy | | | |
| Low baseline | +19.6 months | 0.008 | 0.44 |
| monocytes | | | |
| <5 years since | +4.8 months | 0.025 | 0.62 |
| diagnosis | | | |

In the multivariate predictive model, 4 patient sub-groups (high neutrophil to lymphocyte ratio (NLR), no prior taxane treatment, low monocytes, and <5 yrs since diagnosis) were significant for improved OS.

Immuno-monitoring was also conducted. Blood cell subsets (CD4, CD8, PBMCs, monocytes) and Th1 biomarker (CXCL-10) were measured centrally. Comparison was done using 2-sided Wilcoxon test.

On treatment fold-changes of monocytes (5.81 vs. 2.29; p=0.025), PBMCs (2.00 vs. 1.41; p=0.041), T cells (2.28 vs. 1.48; p=0.086), and CXCL10 (2.78 vs. 1.56; p=0.06) are significantly higher compared to placebo and linked to improved OS. Post baseline CD4 (median 896/μl vs. 736 μl; p=0.038) and CD8 (median 377/μl vs. 223 μl; p=0.005) T cells count increased significantly in patients with improved OS in the IMP321 group vs. placebo.

IMP321 added to paclitaxel elicits significant effects on primary (monocytes) and secondary (CD4; CD8) target cells and associated Th1 biomarker which is significantly associated with improved OS.

### Conclusion

These meaningful gains in OS in certain patient subgroups, representing a meaningful percentage of the overall patient population, are surprising and unexpected as there has been no improvement in recent years in terms of treatment options for HR⁺/HER2-metastatic breast cancer patients eligible to receive chemotherapy (i.e. following endocrine therapy with/without treatment with a CDK4/6 inhibitor). Furthermore, there are no active immunotherapies currently approved or in late-stage trials for this indication. This is because of the non-immunogenic nature of these types of tumours, meaning they are typically less responsive or non-responsive to traditional immunotherapy options.

### Example 2—Active Immunotherapy Paclitaxel-003 (AIPAC003) in HER2-Neg/Low Metastatic Breast Cancer (MBC)

This example describes a planned randomized, double-blind, placebo-controlled Phase 3 trial testing eftilagimod alpha (soluble LAG-3; IMP321) in HER2-neg/low metastatic breast cancer patients receiving paclitaxel, following an open-label dose optimization.

Breast cancer (BC) is the most commonly diagnosed cancer worldwide with an estimated 2.3 million new breast cancer cases reported in 2020 representing 11.7% of all cancer cases. In the United States, the number of estimated new cases for 2020 is 253,465 against 531 086 in Europe, and estimated deaths are 42,617, i.e., 6.2% of all cancer deaths in that year, against 141 765 in Europe.

Breast cancer is the leading cause of death worldwide in the female only population with 685,000 deaths (5th leading cause of cancer death in both sexes). This equates to 1 in every 4 cancer cases is the result of breast cancer in women, and 1 in every 6 cancer deaths. Along with earlier detection and improved treatment, the 5-year net survival for BC has increased in recent years. Advanced/metastatic BC remains a virtually incurable disease, with a median OS of about 3 years and a 5-year survival rate of around 25%.

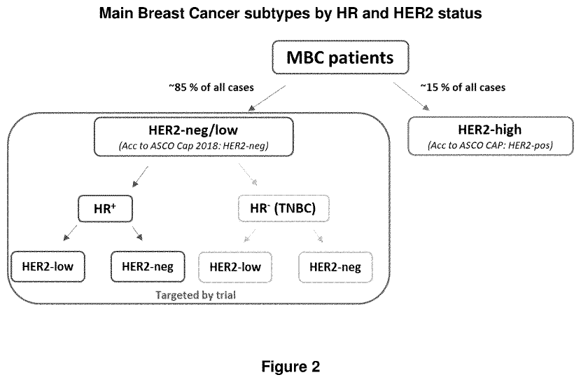

MBC patients are differentiated into four main molecular subtypes based on the degree of expression of HER2 and HR status as depicted in FIG. 2. This trial aims to recruit patients with HR⁺ or HR⁻ and HER2-neg/low MBC. The majority of BC cases (68%) are HR⁺ i.e. positive for the estrogen receptor and/or progesterone receptor, but HER2-neg/low. The 5-year survival rate is estimated at 30%.

HER2-high (previously HER2-pos) expression is defined as a 3+ score by immunohistochemistry (IHC) and/or a positive in situ hybridization (ISH) result (including 2+ on IHC and positive on ISH). All other patients are considered to be HER2-neg/low (Society, A. C. Last Revised: Aug. 25, 2022. Breast Cancer HER2 Status). The HER2-low BC represents a recently proposed classification of the HER2 subtype. Approximately half of all patients with breast cancer have tumors that are HER2-low. They have previously been classified as HER2-neg without effective treatment options with HER2-targeted medicines. HER2-low BC is more common in patients with HR⁺ breast cancer than in those with TNBC. Metastatic triple negative breast cancer (TNBC) is an aggressive disease with poor outcomes and constitutes about 10-20% of all breast cancers cases. This type of breast cancer is characterized by the lack of expression of estrogen receptor (ER), progesterone receptor (PR) and HER2, and the presence of high histologic grades and mitotic rates. Expected 5 years survival is estimated at less than 15%.

This study targets all patients with MBC who are either HR⁺ and HER2-neg/low or TNBC and have received certain therapies before.

### HR⁺ HER2-Neg/Low

The treatment landscape for HR⁺ HER2-neg/low MBC has undergone major changes in recent years. In the past, deprivation of estrogen signaling through single-agent ET was the mainstay of first-line treatment. However, ET resistance eventually occurs in almost every patient, with disease progression typically after 1 year of first-line single agent ET. This treatment paradigm changed with the approval of CDK4/6 inhibitors. In 2015, palbociclib was the first CDK4/6 inhibitor to be approved. By now multiple CDK4/6 based drugs are approved by FDA, EMA, and other health agencies in combination with different types of ET.

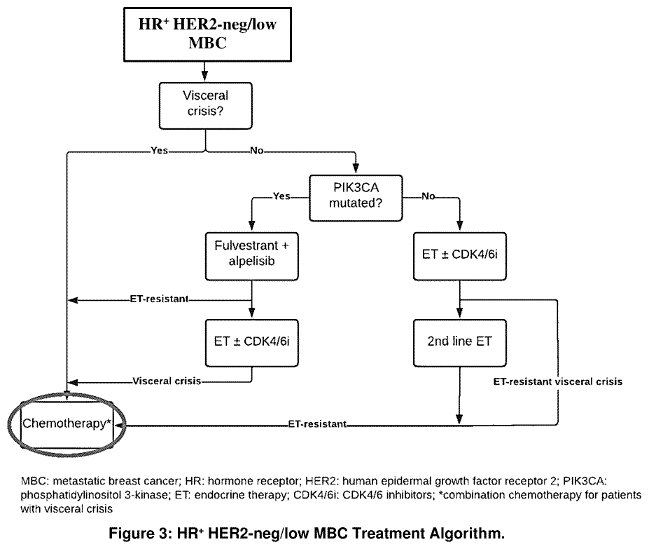

Early after the introduction of CDK4/6 inhibitors, HR⁺ HER2-neg/low MBC patients would in the first line setting typically receive ET alone or in combination with CDK4/6 inhibitors, potentially followed by later endocrine treatment lines (FIG. 3). Chemotherapy was given if ET was not possible, e.g., in case of acute life-threatening disease or endocrine resistance.

Today, the use of CDK4/6 inhibitors has broadened. Preferred regimens according to the NCCN/ESMO guidelines for include CDK4/6 inhibitors with an aromatase inhibitor (AI), fulvestrant±CDK4/6 inhibitor, fulvestrant with a nonsteroidal AI (category 1) and single agent ETs (category 2A). Preferred regimens for second or later lines include fulvestrant+CDK4/6. For patients with a PIK3 mutation there is the option to receive a PI3kinase inhibitor as depicted in FIG. 3. Still, many patients become endocrine resistant at some point, they require further treatment. Single agent chemotherapy and then especially taxanes are commonly used for this patient population (circle in FIG. 3).

Efficacy of Single Agent Chemotherapy for Patients with HR⁺ HER2-Neg/Low after Failure of ET Based Therapy

| | | PFS | |
|---|---|---|---|
| Treatment | ORR (%) | (months) | OS (months) |
| Capecitabine (No prior CDK4/6) | 19-30 | 2.8-5.9 | 9.3-18 |
| Paclitaxel (No prior CDK4/6) | 21-51 | 5.9-8.8 | 20.4-25.8 |
| Paclitaxel (Prior CDK4/6) | 36 | 6.7 | 14.9 |

ORR: objective response rate;
PFS: progression-free survival;
OS: overall survival

The addition of CDK4/6 inhibitors has increased PFS/OS for ET based therapies dramatically, but it was shown in different studies that post CDK4/6 therapy overall survival of chemotherapy decreased. In the AIPAC study OS in paclitaxel plus placebo group dropped from 20.4 months to 14.9 months (in case patients were pre-treated prior with CDK4/6 compared to the ones with no prior CDK4/6 therapy). Independent of other poor prognostic marker prior CDK4/6 had a negative impact on PFS and OS.

Generally, it can be expected that patients receiving chemotherapy nowadays have received many more lines of therapy and are in a later stage (more metastatic sites, more involvement of liver and other distant metastases) compared to 2015 and before.

### TNBC

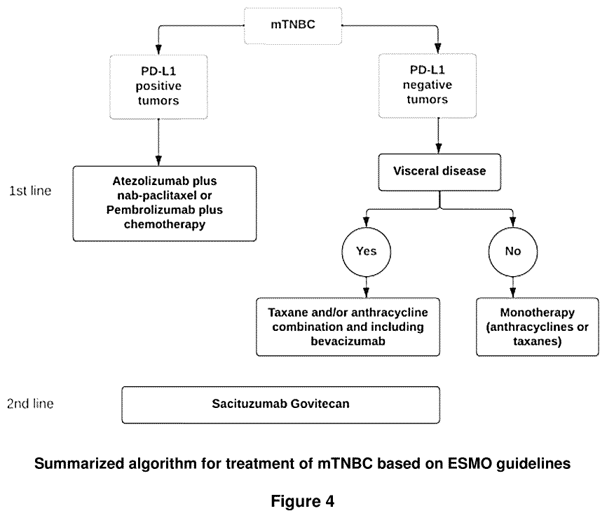

For TNBC patients who present with metastatic disease the choice of therapy depends predominantly on PD-L1 expression as presented in FIG. 4. For patients with PD-L1-positive tumors (expressed in 38% of TNBC according to the trial Keynote-355), anti-PD-1 plus chemotherapy is advised.

The FDA and EMA approved atezolizumab in combination nab-paclitaxel for the treatment of adult patients with unresectable locally advanced or metastatic PD-L1 positive TNBC in 2019, based on the results of the Phase 3 IMpassion130 trial (cut-off April 2018). The indication was then withdrawn in 2021 in the US following the results from the phase 3 IMpassion131 trial, in which the PD-L1 inhibitor plus paclitaxel did not demonstrate a statistically significant improvement in PFS over paclitaxel alone.

In 2021, both EMA and FDA granted approval to pembrolizumab in combination with chemotherapy (paclitaxel, nab-paclitaxel, or carboplatin-gemcitabine) for patients with locally recurrent unresectable or metastatic TNBC whose tumors express PD-L1 (Combined Positive Score (CPS)≥10). The approval was based on KEYNOTE-355, a multicenter, double-blind, randomized, placebo-controlled trial in patients with locally recurrent unresectable or metastatic TNBC, who had not been previously treated with chemotherapy in the metastatic setting. Among patients with CPS of 10 or more, median PFS was 9.7 months in pembrolizumab-chemotherapy group and 5.6 months in placebo-chemotherapy group (cut-off, Dec. 11, 2019).

For patients not eligible for anti PD-1/PD-L1 containing therapy (˜62%) chemotherapy remains the primary systemic treatment, with international guidelines supporting the use of single-agent taxanes (with or without bevacizumab) or anthracyclines as first-line therapy, with median OS up to 18 months. Thus, patients with PD-L1-negative tumor or after failure of anti-PD-1/PD-L1 based therapy have a limited choice of treatment.

### Efficacy of Selected Treatment Option for Metastatic TNBC Patients not Eligible for PD-1/PD-L1 Based Therapy.

| | | PFS | OS |
|---|---|---|---|
| Treatment | ORR (%) | (months) | (months) |
| Taxane monotherapy | 23-46 | 4-5 | 12-18 |
| Taxane + bevacizumab | 49 | 7.2 | 18.3 |

Although improvements have been made in the adjuvant treatment of early breast cancer over the last decades, ˜30% of patients initially diagnosed with regional stage disease will eventually experience recurrence in the form of metastatic breast cancer (MBC). Additionally, 5-10% of patients may also be diagnosed with metastatic disease at the time of first diagnosis. Despite the major improvements for early and late-stage BC patients with the introduction of new therapies, no improvements were made for patients when they become eligible for chemotherapy with metastatic disease.

Today, HR⁺ HER2-neg/low MBC patients receive chemotherapy later in the course of their disease and are more heavily pre-treated at the time they start chemotherapy (FIG. 3). Also, patients with HR⁺ MBC who have received several treatments acquire a more aggressive clinical behavior and will ultimately become refractory to any intervention. In the PALOMA-3 trial of palbociclib+fulvestrant, duration of post-trial chemotherapy duration was reduced from 6 to 5 months which is a decrease of about 20% (Turner, et al., 2018. Overall Survival with Palbociclib and Fulvestrant in Advanced Breast Cancer. N Engl J Med 379:1926-1936). In a multivariate analysis in AIPAC, prior CDK4/6 treatment was an independent poor prognostic marker, with a HR of 1.374 for OS and HR of 1.65 for PFS. The observed median OS in AIPAC in patients receiving paclitaxel plus placebo is clearly lower (median 14.9 months) in patients pre-treated with CDK4/6 compared to historical data with median OS of ˜24 months for patients receiving single agent chemotherapy for MBC.

Regarding TNBC, novel therapies are urgently needed given the suboptimal outcomes of chemotherapy alone and the lack of treatment, especially in patients not eligible for anti PD-1/PD-L1 therapies (see FIG. 4). Furthermore, no active immune-oncology (IO) treatment is approved for this patient population.

In conclusion, there remains a high unmet medical need with median OS in the range of 12-18 months for HR⁺ HER2-neg/low MBC patients who have exhausted endocrine based treatment options or TNBC patients who are ineligible for anti-PD-L1/PD-1 based therapy, and thus have become candidates for chemotherapy such as weekly paclitaxel.

### Study Design

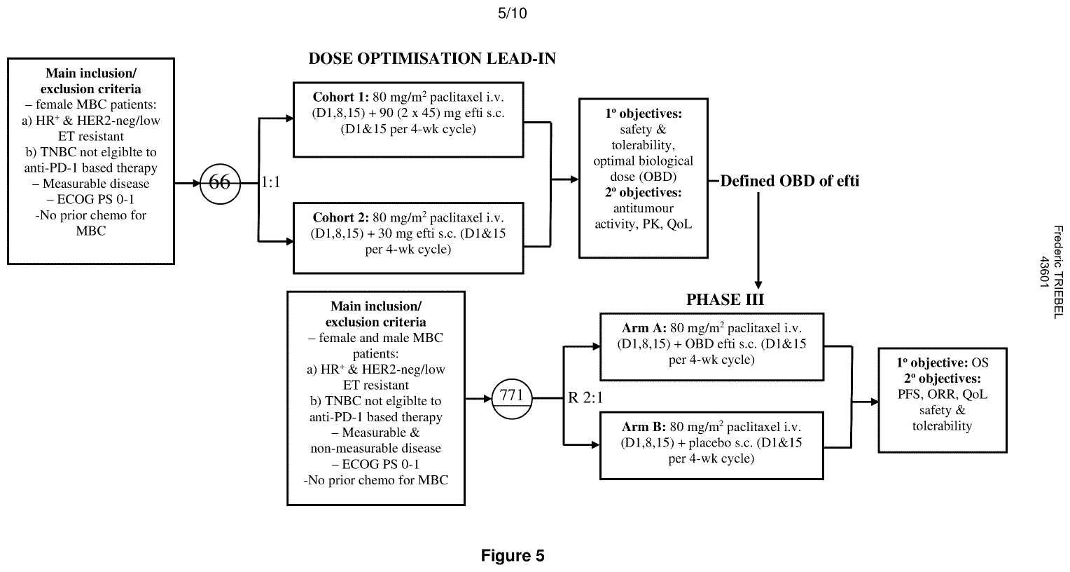

This trial consists of an open-label dose optimization lead-in component followed by a double-blinded, Randomized, placebo-controlled Phase 3 component as displayed in FIG. 5.

The dose-optimization lead-in phase comprises two parts: a safety lead-in followed by the randomized dose optimization lead-in.

### Dose Optimization Lead-In Component

In the initial open label lead-in component, the optimal biological dose (OBD) of efti in combination with weekly paclitaxel will be determined based on the following parameters:
Safety 1 and 2: % of patients with DLTs and % of patients with adverse reactions leading to permanent discontinuation of efti within the first 4 cycles (other than hypersensitivity to paclitaxel at C1D1).
Tolerability: % of patients with any injection site reaction of any grade lasting >3 days and % of patients with any injection site reaction of grade 3 or higher severity of any duration.
Efficacy: % of patients with BOR of PR/CR as per RECIST 1.1.
PDMs 1 and 2: % of patients with a 1.4-fold increase in each of the PDMs (PDM1—peripheral CD8+ T cells and PDM2—absolute lymphocyte count) compared to baseline within the first 4 cycles.

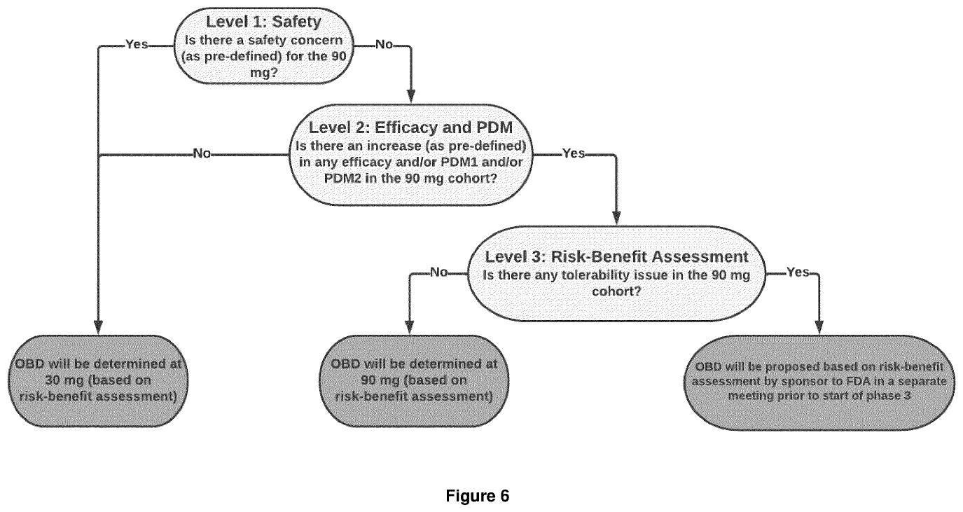

The OBD will be decided based on the data for both dose levels once at least 29 evaluable patients per dose cohort have been randomized. The decision algorithm will be conducted at three different levels as outlined in FIG. 6.

### Efficacy: Best Overall Response (BOR)

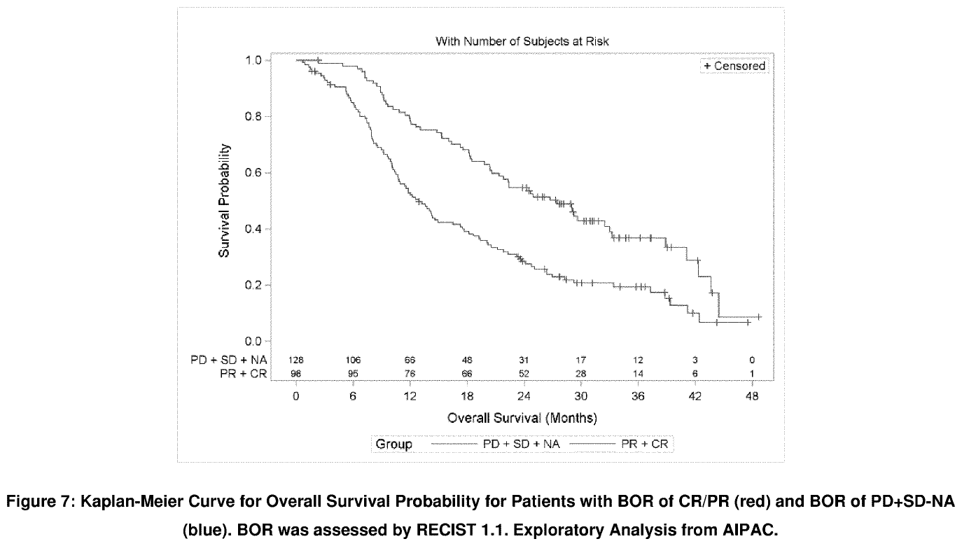

Overall response rate (ORR) was positively correlated with improved overall survival in the AIPAC study as depicted below. Patients with a BOR of PR or CR according to RECIST 1.1 have a higher likelihood of an overall survival >median. The Kaplan Meier-plots are significantly different (see FIG. 7). Median decreases significantly (p<0.001) from 27.5 [95% CI 20.6; 33.0] to 12.9 (95% CI 10.7-16.5) for patients who did not have a PR/CR as BOR. The HR is 0.5 [95% CI 0.37; 0.69] with a p-value <0.001 favoring a BOR of PR/CR making ORR a good surrogate endpoint for efficacy (OS which is primary in the Phase 3 part) of this combination and was hence selected as the efficacy marker.

### PDM1—Absolute Number of CD8⁺ T Cells

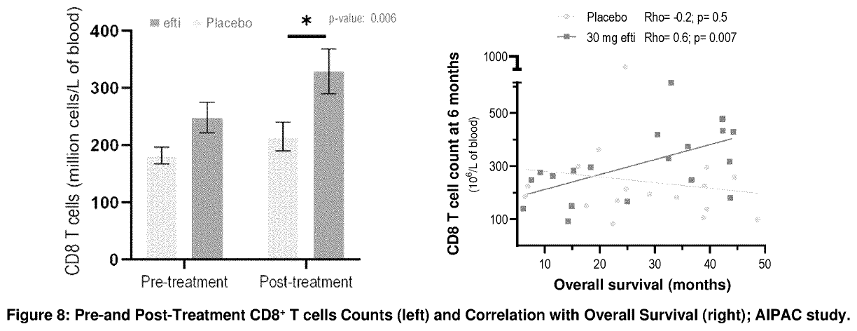

In the AIPAC study, in patients where fresh whole blood was directly analyzed ex vivo using FACS (n=75), a sustainable, statistically significant increase in circulating CD8⁺ T cells was observed in the efti arm, but not in the placebo arm, and was found to be correlated with OS (FIG. 8). This effect was seen in samples taken on treatment, in the efti arm only; of note, samples were taken 2 weeks after the previous efti dose (note that efti disappears from the bloodstream 3-4 days after injection), hence, showing the minimal residual effect of efti on study, just before the next efti injection. On the other hand, pre-study levels of CD8+ T cells were not correlated with OS in either treatment arm. Efti increases secondary target cells (CD8+ T cells) in the peripheral blood, leading to improved OS. Based on the above-mentioned reasons the increase in circulating CD8+ T cells is a good surrogate for efti activity, was positively correlated with overall survival and can be measured in a standardized way and was hence selected as PDM1.

### PDM2—Absolute Lymphocyte Count (ALC)

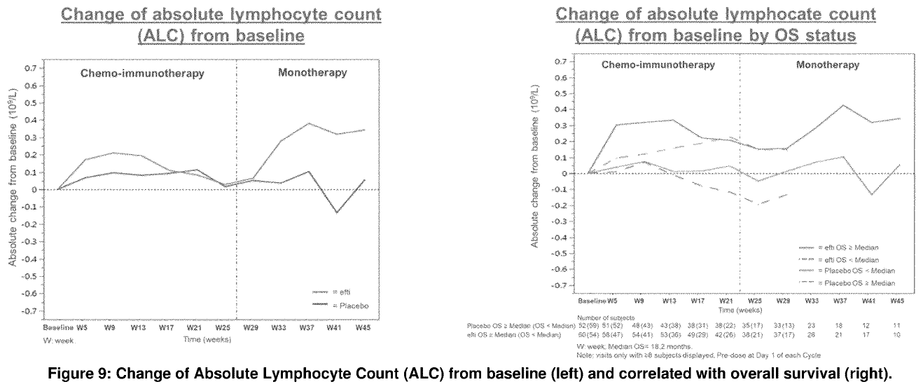

The absolute lymphocyte count showed an early (after 4 weeks) and sustainable increase (≥0.2/nl) within the efti arm (green line FIG. 9). Increase of ALC is linked to improved survival for patients treated with efti arm, but not for patients in the placebo arm (FIG. 9). A little more than half of the patients in AIPAC had an absolute increase of ≥0.2/nl which leaves room for improvement in the 90 mg cohort. As this increase is observed early, it could be used in the future as a potential early on study biomarker for treatment continuation. Based on the above-mentioned reasons the absolute increase of lymphocytes is a good surrogate for efti activity, was positively correlated with overall survival and can be measured in a standardized way and was hence selected as PDM2.

### Phase 3 Component

In the Phase 3 component of the trial, approximately 771 HER2-neg/low MBC patients will be randomized 2:1 to Arm A (active arm): paclitaxel+efti at OBD and Arm B (control arm): paclitaxel+placebo. Hence, there is a 66% chance for patients to be randomized to the active treatment arm and all patients will receive weekly paclitaxel as standard of care. In this trial, the use of a placebo associated with double blinding greatly improve the quality of the data and its interpretation.

Patients will be stratified by (1) type of MBC (HR⁺ vs- HR⁻), (2) prior CDK4/6 (yes versus no) and age (<65 years and >65 years). The trial is event-driven and will be considered complete after recording the required number of events.

### Schedule in Dose Optimization Lead-In and Phase 3

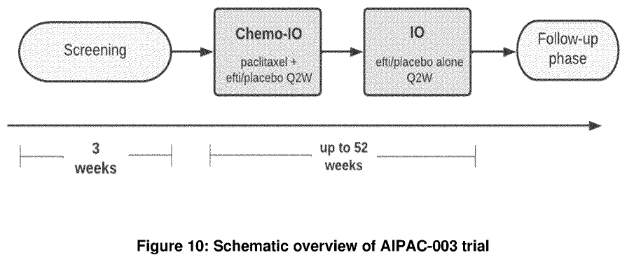

Each patient will undergo a screening period of up to 3 weeks. Treatment for the dose optimization lead-in and the Phase 3 components of the trial will consist of a chemo-immunotherapy (chemo-IO) phase followed by an immunotherapy (IO)-phase (FIG. 10). The chemo-IO phase consists of 6 cycles of 4 weeks (28 days) each. Six (6) cycles of paclitaxel are planned. In case paclitaxel is well tolerated, patients can continue beyond 6 cycles at the discretion of the investigator based on the individual tolerability of the patient. In case paclitaxel needs to be stopped due to toxicity prior to completion of 6 cycles, patients are allowed to move on to efti/placebo alone (IO-phase) in case 4 cycles with paclitaxel were completed.

During each of the chemo-IO cycles, patients will receive 80 mg/m² paclitaxel intravenously on D1, D8 and D15 in both treatment arms in a 28-day (4-week) cycle, followed by 30 mg or 90 mg efti in the dose optimization lead-in, and placebo or the OBD of efti in the Phase 3 component, subcutaneously on D1 and D15 in a 28-day (4-week) cycle. The IO-phase is planned to start at cycle 7 with efti or placebo (the latter applies only in Arm B in Phase 3) administered s.c. on Day 1 and 15 of each cycle. The IO-Phase could start earlier or later depending on paclitaxel tolerance of each individual patient. A maximum of 13 cycles (approximately 12 months) of treatment are planned. A schematic overview of the screening, treatment and follow-up period is shown in FIG. 10.

Patients will stay on treatment until disease progression, unacceptable toxicity, completion of the trial treatment or discontinuation for any other reason. The treatment period concludes with an end of treatment (EOT) visit 1-3 weeks after last trial drug administration. Follow-up for progression (until disease progression in case patient did not show disease progression at EOT) and survival (until death for every patient) will be performed until the end of the trial, withdrawal of consent or loss to follow-up, whichever occurs first.

Radiological assessment will be performed at intervals of 8 weeks until week 32, and every 12 weeks thereafter. They will be evaluated according to Response Evaluation Criteria in Solid Tumors (RECIST) version 1.1 at the clinical sites (investigator read) and treatment decisions will be based on investigator read.

### Justification for 30 mg Dose of Eftilagimod Alpha (Efti)

The desired PD effects in vitro and ex vivo, i.e., activation of primary and secondary immune cells as exemplified by the induction of cytokines, is observed starting at efti concentrations of about 1-10 ng/ml. In preclinical tumor efficacy models, the combination of efti with chemotherapy or PD-1 antagonists showed synergistic effects at dose levels corresponding to an estimated plasma Cavg24h in the range of 2-5 ng/ml. Thus, the in vitro effective concentration range also seems to be effective in proof-of-concept animal studies. Available clinical data show that at a dose of 30 mg s.c. to cancer patients, a comparable Cavg24h of 3.3 ng/ml is achieved. At a dose of 6 mg, a lower Cavg24h of 0.8 ng/ml was observed.

Efti doses of 6 and 30 mg given clinically lead to plasma Cmax of ≥1 ng/ml in most patients, this concentration reflecting the minimum concentration for desired efti PD effects. However, systemic efti levels of >1 ng/ml and lasting for ≥24 h have been observed in about 90% of patients receiving efti 30 mg, compared to only 20% at 6 mg.

PD markers related to T cell immune response, such as CD4+ and CD8+ T cells numbers and activation status as well as relevant T cell cytokines like IFN-γ and CXCL10, showed meaningful changes in patients after dosing with efti every 2 weeks, however the proportion of patients with upregulation of at least one of the aforementioned markers (defined as 1.4-fold increase compared to baseline) increased from about 50% of patients at 1 mg to about 90% at 30 mg. 30 mg is considered a safe and tolerable dose. The 30 mg dose has been tested widely in NSCLC, HNSCC, melanoma and HR+ MBC in combination with pembrolizumab or weekly paclitaxel showing encouraging efficacy compared to historical controls.

### Justification of 90 mg Dose of Eftilagimod Alpha (Efti)

While it is recognized that 30 mg efti is an active dose, based on the excellent safety profile and the limited effects on PFS, investigation of a higher dose in combination with weekly paclitaxel will be investigated. The 90 mg efti dose was selected based on PK simulation carried out on 3 different dose levels (30 mg, 60 mg and 90 mg) and on various parameters (e.g., Cmax, AUC0-48h and Cavg24h) using the available popPK model. AUC0-48h and Cavg24h 75% percentile for the 30 mg does not overlap with the 25% percentile of the 90 mg ensuring that at least 50% of the patients are expected to have a higher AUC0-48h and Cavg24h in the 90 mg group compared to the 30 mg group. Distribution of simulated concentration-to-time profiles at 60 mg and 90 mg as compared to 30 mg led to the same conclusion. Median Cmax is expected in the range of ˜15 ng/ml for the 90 mg dose. In the available PK database, there are 5 patients with a Cmax of >10 ng/ml at dose 1 and there was no safety concern identified in these patients, i.e., no grade ≥3 adverse events related to efti, 3 out of the 5 patients reported in total 8 treatment emergent adverse events with onset date after first and before second dose, all of which were assessed by investigator as related to study treatment. These adverse reactions (AR) were mild (62.5%) or moderate (37.5%) in severity, all but one recovered completely, and no action were taken with study treatments due to these events. In the nonclinical toxicity study the NOAEL dose was defined with 15 mg/kg, to which the 90 mg dose would correspond to, based on a 60 kg body weight patient.

The fact that the 90 mg dose will be administered in 2 separate injections of 45 mg (1.8 ml) each further mitigate any potential tolerability risks. The rationale for this is the volume limitation i.e., subcutaneous injection volumes larger than 2 mL are associated with various issues including injection pain, adverse events at the injection site, and injection site leakage (i.e., backflow of injected solution). Given that the 2 injections will be administered max. 15 min apart no impact on the PK profile is expected compared to a single injection of 30 mg (1.2 ml).

Overall, a flat dosing of efti for the dose optimization lead-in appears reasonable for an immunostimulant with a wide therapeutic index.

### Justification for the Route and Frequency of Eftilagimod Alpha (Efti)

Route of administration for efti is subcutaneous. The primary target cells i.e., dendritic cells are abundant in the subcutaneous tissue. The choice of the administration route was based on the rationale that efti, as an agonist, is to be delivered to its cellular targets at a low concentration for an extended period (at least for 24 hours) rather than reaching a rapid peak in exposure which would occur after intravenous (i.v.) administration with risk of proinflammatory systemic events.

The rationale for dosing frequency is that efti, being an agonist, will stimulate immune modulators in a sustained manner over several months. This steady activation of effector T cells is desired without impact on tolerability. It is known that secondary T cell responses to antigenic peptides demonstrate rapid kinetics with peak CD8+ T cell proliferation at Day 7 in vitro, and that activated CD8+ T cells need to rest for another week to be able to respond again to a new antigenic stimulus. Overall, a dosing schedule of every 2 weeks is considered the best and most intense administration schedule for patients with advanced or metastatic cancer in need for an effective immune stimulation without induction of safety or local tolerability concern.

In combination with anti-PD-1 and anti-PD-L1 therapies efti is given on the same day without any additional safety observations (IB ed 9.0). Based on this experience and to limit the number of days on site for each patient (3 vs. 5 for every 28-day schedule) the schedule applied in AIPAC was adapted and efti will be administered ≥30 min after end of paclitaxel infusion on the same day of paclitaxel.

### Justification for Dose of Paclitaxel

Weekly dosing of paclitaxel has been evaluated to increase dose density and improve tolerability. It has been shown in large, randomized studies that weekly paclitaxel is superior in terms of safety and efficacy compared to every 3 weeks application (D'Amico, et al., 2021. Standard of Care in Hormone Receptor—Positive Metastatic Breast Cancer: Can We Improve the Current Regimens or Develop Better Selection Tools? American Society of Clinical Oncology 18:331-334; Huard, et al., 1997. Characterization of the major histocompatibility complex class II binding site on LAG-3 protein. Proc Natl Acad Sci USA 94:5744-5749). Weekly administration of paclitaxel 80 or 90 mg/m² is possible; an 80 mg/m² dose will be used in AIPAC-003, as data on the combination with efti are already available from AIPAC, and because this dose is considered to have a slightly more favorable safety profile than 90 mg/m². Paclitaxel 80 mg/m² monotherapy given on days 1, 8 and 15 of a 28-day cycle is a widely used regimen for treatment of metastatic breast cancer (Decker, et al., 2017. A randomized phase II study of paclitaxel alone versus paclitaxel plus sorafenib in second- and third-line treatment of patients with HER2-negative metastatic breast cancer (PASO). BMC Cancer 17:499; Hernandez-Aya and Ma. 2016. Chemotherapy principles of managing stage IV breast cancer in the United States. Chinese Clinical Oncology 5:42; Keane, M. 2021. PACLitaxel 80 mg/m2 Day 1, 8 and 15 Monotherapy-28 Day. 1 ed. NCCP, H-S-E homepage, National Cancer Control Programme; 2022. Regimen Reference Order—BRST—PACLitaxel Metastatic every 7 days. CancerCare Manitoba, CancerCare Manitoba webpage. 2).

Patients will need to meet the following criteria (inter alia) if they are to take part in the trial:

1. Metastatic HR⁺ (estrogen receptor positive and/or progesterone receptor positive) or hormone receptor negative (HR−), and HER2-neg/low breast adenocarcinoma, histologically proven by biopsy on the last available tumor tissue (primary tumor and/or a metastasis; metastasis preferred). Note: estrogen and/or progesterone receptor positivity is defined as ≥1%, HER2 receptor negativity is defined in line with ASCO/CAP guidelines (Burstein, et al. 2021. Endocrine Treatment and Targeted Therapy for Hormone Receptor-Positive, Human Epidermal Growth Factor Receptor 2-Negative Metastatic Breast Cancer: ASCO Guideline Update. J Clin Oncol: Jco2101392; Wolff, et al. 2013. Recommendations for human epidermal growth factor receptor 2 testing in breast cancer: American Society of Clinical Oncology/College of American Pathologists clinical practice guideline update. J Clin Oncol 31:3997-4013.). This HER2-low category includes those who have IHC scores of 1+ and 2+ without amplification (as measured by that ISH test). Estrogen-, progesterone-, and HER2 receptor testing is to be conducted in line with regional guidelines (Wolff, A. C. 2018. HER2 Testing in Breast Cancer: American Society of Clinical Oncology/College of American Pathologists Clinical Practice Guideline Focused Update Summary. Journal of Oncology Practice: 14(17): 437-441) (e.g., ASCO/CAP guidelines).

2. Patients with HR⁺ MBC who progressed on or after ≥1 line of endocrine based therapy and are indicated to receive chemotherapy for metastatic disease. Meeting any of below conditions:
a. Primary endocrine resistance: recurrence/relapse ≤2 years after the start of adjuvant endocrine therapy for early breast cancer, or progression within 6 months of 1st line endocrine based therapy for metastatic breast cancer.
b. Secondary endocrine resistance: recurrence/relapse >2 years after starting adjuvant endocrine based therapy, recurrence/relapse <12 months of finishing adjuvant endocrine based therapy or progression after >6 months of endocrine based therapy for metastatic breast cancer.

Note: Prior targeted therapies (e.g., CDK4/6 or mTOR inhibitors) are allowed. Patients with known PI3K mutation may have received PI3K inhibitors prior to inclusion. Patients with a BRCA mutation may have received a PARP inhibitor prior to inclusion. Any treatment given in combination with endocrine therapy as first line treatment for MBC is allowed, apart from traditional, cytotoxic chemotherapy.

3. Patients with TNBC who are indicated to receive paclitaxel chemotherapy without anti-PD-1/PD-L1 therapy in the 1st line setting for metastatic disease. Note: prior anti-PD-1/PD-L1 therapy in the adjuvant setting is allowed. Patients with a BRCA mutation may have received a PARP inhibitor prior to inclusion.

4. Dose optimization lead-in: Female of age 18 years-of-age or older.

Phase 3: Female or male of age 18 years-of-age or older.

5. All patients of childbearing potential must have a negative highly sensitive pregnancy test at screening and agree to use a highly effective method for contraception according to the EU Clinical Trial Facilitation Group guidance from time of trial entry until at least 6 months after the last administration of trial drug. The partners of patients with childbearing potential must also apply contraceptive methods. Patients who are either:
a. Postmenopausal (≥60 years of age, or <60 years of age and amenorrhoeic for 12 months in the absence of chemotherapy, tamoxifen, toremifene, or ovarian suppression with follicle-stimulating hormone (FSH) above 40 U/L and estradiol below 30 ng/L; or if taking tamoxifen or toremifene, and age <60 years, then FSH and estradiol in the postmenopausal range), permanently sterilized (e.g., bilateral tubal occlusion, hysterectomy),
b. Incapable of pregnancy are not considered to be of childbearing potential.

6. Dose optimization lead-in only: evidence of measurable disease as defined by RECIST 1.1. Note: patients with non-measurable disease as defined by RECIST 1.1 may enroll to the Phase 3 component.

7. Laboratory criteria:
a. Total white cell count ≥3×10⁹/L
b. Platelet count ≥100×10⁹/L
c. Hemoglobin ≥9 g/dL or 5.58 mmol/L
d. Absolute Neutrophil Count (ANC) ≥1.5×10⁹/L
e. Serum creatinine clearance by CKD-EPI >30 mL/min
f. Total bilirubin ≤20 μmol/L, except for familial cholemia (Gilbert's disease)
g. Serum ASAT and ALAT≤3 times ULN or ≤5 times ULN if liver metastases are present

Patients are to be excluded from the study for any of the following reasons (inter alia):

1. Prior chemotherapy for metastatic breast adenocarcinoma. Note: only regimens given in the metastatic setting and containing traditional, cytotoxic chemotherapeutic agents are considered as grounds for exclusion. Pre-treatment with any kind of targeted agents can be acceptable if all other applicable criteria are met.

2. Patients with HR⁺ MBC who have received <1 line of ET based therapy in the metastatic setting.

3. Patients with HR⁺ MBC who are not primary or secondary resistant to ET-based therapy and would be candidates to ET based therapy as per applicable treatment guidelines.

4. TNBC patients who are candidates for PD-1/PD-L1 therapy in combination with chemotherapy.

### Trial Treatments

### Identity of Investigational Medicinal Products (Study Agents)

### Eftilagimod Alpha

The efti drug product is a single-use, preservative-free, sterile solution of efti for subcutaneous injections at a concentration of 25±1.2 mg/mL. The drug product is filled into 2 mL glass vials with an extractable fill volume of ≥1.2 mL to be stored at 2° C. to 8° C. and protected from light.

### Matching Placebo to Eftilagimod Alpha

Placebo matching the appearance and injection characteristics of the efti drug product. The placebo is filled into 2 mL glass vials with an extractable fill volume of ≥1.2 mL to be stored at 2 to 8° C. and protected from light.

### Dose Regimen

### Eftilagimod Alpha

Efti will be administered at a dose of 30 mg and 90 mg in the dose optimization lead-in component. Repeated s.c. doses of efti will be administered, on D1 and D15 in a 4-week cycle during the chemo-IO and the IO components. Efti will be administered at least 30 minutes after paclitaxel infusion is complete. The maximum number of efti administrations is 26.

Efti (at the OBD) or placebo will be administered during the Phase 3 component, following the same regimen as in the dose optimization lead-in component.

The route of administration for efti will be s.c. injection. For the 30 mg dose (1.2 mL), a single anatomical site will be used on the anterior face of the thigh. The location of the injection site should be alternated between thighs with each injection (if, for example, on C1D1 the first injection is given into the left thigh, then the next injection on C1D15 will be given into the right thigh etc.).

The 90 mg dose will be administered in two separate injections (timed at a maximum 15 min apart) of 45 mg (1.8 mL) as follows: a single anatomical site will be used on the anterior face of the one thigh and a single anatomical site in the anterior face of the other thigh.

The injection(s) should be performed slowly to avoid discomfort at the site of injection.

### Paclitaxel

Paclitaxel will be administered as a 1-hour intravenous (i.v.) infusion of 80 mg/m² (with a maximum of 160 mg for patients with body surface area of ≥2 m2) on D1, D8, and D15 of a 4-week cycle.

The method of administration and supportive care will follow local clinical practice, apart from corticosteroid premedication, for which the following restrictions apply. On D1 and D8 of the first treatment cycle (i.e., before the first two paclitaxel infusions), 10 mg dexamethasone i.v. is to be given only once, 30 minutes prior to paclitaxel infusion. Oral corticosteroid premedication or corticosteroid premedication initiated earlier than specified is not allowed. No further premedication with corticosteroids should be given if the first 2 i.v. infusions of paclitaxel (1 cycle) have been well tolerated. In patients who develop a reaction to paclitaxel, for all following paclitaxel administrations, 10 mg dexamethasone (or an equivalent dose of other systemic corticosteroid) is the maximum dose for premedication.

There are no restrictions to other means of premedication for paclitaxel, for any of the treatment cycles, including but not limited to antihistamines.

Six (6) cycles of paclitaxel are planned. In case paclitaxel is well tolerated, patients can continue beyond 6 cycles at the discretion of the investigator based on the individual tolerability of the patient.

In case paclitaxel needs to be stopped due to toxicity prior to completion of 6 cycles, patients are allowed to move on to efti/placebo alone (IO-phase) in case 4 cycles with paclitaxel were completed.

A patient will stay on treatment until disease progression, unacceptable toxicity, death completion of the trial treatment or discontinuation for any other reason.

A commercially available paclitaxel product will be used. Formulations other than the conventional ones, e.g., paclitaxel medicines formulated as albumin-bound nanoparticles (nab-paclitaxel) are strictly not allowed in the trial.

### Study Assessments

### Efficacy Assessments

### Radiological Scans and Color Digital Photography

A computed tomography (CT) scan with contrast of the chest, abdomen and pelvis is required for each patient at each time point. Tumor imaging is strongly preferred to be acquired by CT with contrast enhancement. For the abdomen and pelvis, contrast-enhanced magnetic resonance imaging (MRI) may be used when CT with iodinated contrast is contraindicated, or when mandated by local practice and for the chest, a non-contrast CT of the chest is recommended to evaluate the lung parenchyma.

If brain imaging is performed to document the stability of existing metastases or to exclude/confirm suspected new brain metastases during the trial course, MRI is the strongly preferred modality (not mandatory for patients without evidence of brain disease). If brain MRI is contraindicated for any reason, head CT including the brain should be performed instead. The same imaging technique regarding modality, ideally the same scanner, and the use of contrast should be used in a patient throughout the trial to optimize the reproducibility of the assessment of existing and new tumor burden and improve the accuracy of the assessment of response or progression based on imaging (see Imaging Acquisition Guidelines).

MRI scans of the head and color digital photography of skin lesions will be performed as clinically indicated.

MRI examinations should be performed with a 1.5 T or 3 T MRI system.

The machines (CT or MRI) to be used are described in the imaging manual. The process for image collection and transmission to the central imaging vendor can be found in Imaging Acquisition Guidelines.

A lesion identified at follow-up in an anatomical location that was not scanned at screening is considered a new lesion and will be handled accordingly. An example of this is when a patient has visceral disease at screening and while on trial requires a CT or MRI scan of the brain which reveals metastases. The patient's brain metastases are considered new lesions even if there was no brain imaging conducted at screening.

Patients with symptomatic cerebral and/or leptomeningeal metastases are not allowed to enter the study. For all other patients, study-specific radiological scans of the brain are not foreseen, except for patients with abnormalities during a neurological screening assessment; it is at the discretion of the Investigator.

Skin lesions if present at screening will be assessed in the same intervals as described above. During every time point scheduled for radiological assessment, if any skin lesion is present, color digital photography will be performed.

All treatment decisions will be based on Investigator's read. Images will be collected for retrospective blinded independent central read assessment.

### Imaging

Imaging will be performed to determine tumor burden according to following schedule:
Screening (Lead-in and Phase 3): ≤21 days prior to initiation of therapy, historical images (obtained within a window of 6 weeks prior to the start of study treatment) can be used for assessment of patient eligibility, if they are in line with the imaging protocol described in the Imaging Acquisition Guidelines.
For patients in the Lead-in and Phase 3 components radiological assessments carried out according to RECIST 1.1 should be performed prior to receiving any treatment during the screening and every 8 weeks until week 32, every 12 weeks thereafter. It may be performed±3 days prior to that specific radiological assessment until week 32. The imaging schedule is not affected by the treatment and starts from the day of randomization.
Actions taken with the study treatment/study agent (e.g., treatment delayed) do not affect the imaging schedule. The imaging schedule shall remain as determined from randomization.

Clinical Lesions: Clinical lesions detected by physical examination will only be considered measurable if they are superficial and ≥10 mm in diameter as measured with calipers (for example skin nodules). If skin lesions are present at screening, the documentation of these visible lesions (e.g., index tumor lesion and/or new skin lesions) by color photography including a centimeter ruler to estimate the size of the lesion is recommended. All photographs will maintain the anonymity of the patient and will be labelled using the patient's study identifier and photograph date.

If skin lesions can be evaluated by both, clinical examination and imaging, imaging evaluation should be undertaken since it provides a more objective evaluation.

Superficial clinical lesion (e.g., skin nodule) must be measured in at least one dimension (longest diameter in the plane of measurement will be recorded) with a minimum size of the longest axis being ≥10 mm as imaged with scale in color photography per RECIST 1.1.

If a patient has clinically indicated skin lesion(s), the investigator site will perform color digital photography of all skin lesions using a ruler held flush to the skin next to the longest diameter of the lesion to indicate the size of the lesion at every time point scheduled for radiological scans that a lesion is present. Once a lesion(s) is documented, the target area should be documented at every subsequent time point for the duration of the study.

A lesion identified at follow-up in an anatomical location that was not scanned at baseline is considered a new lesion and will indicate disease progression. An example of this is when a patient has visceral disease at baseline and while on study requires a CT or MRI scan of the brain which reveals metastases. The patient's brain metastases are considered as evidence of PD even if there was no brain imaging conducted at baseline. Furthermore, patients who require bone scans will undergo nuclear medicine-based bone scans (e.g., 99mTc-MDP bone scans). All single new foci of radiotracer uptake will be confirmed either by CT, MRI, or biopsy to prove progression. The presence of two or more new foci in the absence of a benign cause (e.g., fracture or trauma) are considered to as evidence of PD even if there was no bone imaging at baseline.

### Progression-Free Survival

PFS is defined as the number of days between the date of treatment assignment (lead-in component) or the time from randomization (Phase 3 component) to documented disease progression or death from any cause as assessed by the investigator assessment based on RECIST 1.1. The date of disease progression or censoring for PFS will be determined according to the conventions listed below. These conventions are based on the December 2018 FDA Guidance for Industry, ‘Clinical Study Endpoints for the Approval of Cancer Drugs and Biologics’ (http://www.fda/media/71195/download) and on the April 2015 FDA Guidance for the Industry, ‘Clinical Trial Endpoints for the Approval of Non-Small Cell Lung Cancer Drugs and Biologics’ (https://www.fda.gov/media/116860/download).

| Situation | Date of Progression or Censoring | Outcome |
|---|---|---|
| Death or disease progression | Date of death or first radiological | Progressed |
| between planned radiological | assessment showing disease | |
| assessments | progression, whichever occurs first | |
| Death before first radiological | Date of death | Progressed |
| assessment | | |
| No baseline or post-baseline | Start date of treatment with the study | Censored |
| radiological assessments | drug | |
| New anticancer treatment started | Date of last radiological assessment | Censored |
| before documentation of disease | prior to the start of non-protocol | |
| progression or death | anticancer treatment | |
| Death or progression after one | Date of missed radiological | Progressed |
| missed radiological assessment | assessment visit | |
| Death or progression after more | Date of last radiological assessment | Censored |
| than one missed radiological | visit without documentation of disease | |
| assessment | progression that is before the missed | |
| | visit | |
| Alive and without documentation | Date of last radiological assessment | Censored |
| of disease progression | | |

### Overall Survival

Overall survival (OS) is defined as the time between the date of treatment assignment (lead-in component) or as time from randomization to death from any cause.

Patients who are lost to follow-up and those who are alive at the date of data cutoff will be censored at the last date the patient was last known alive, or date of data cutoff, whichever occurs first.

The overall survival follow-up visit should be performed every 12 weeks after enrollment into the trial for the first 2 years and every 24 (+/−4 weeks) weeks thereafter. The visit can be performed via a telephone call. Additionally, any next line of anti-cancer therapy will be recorded. If necessary, patients may be contacted occasionally outside of this FU window. OS-FU is to be conducted until trial end, death, withdrawal of consent or lost to follow-up, whichever occurs first.

### Tumor Response

Only patients with measurable disease at screening will be included in the lead-in component of the study. Patients with non-measurable disease may be enrolled in the Phase 3 component of the trial.

When more than one measurable lesion is present at baseline all lesions up to a maximum of five lesions total (and a maximum of two lesions per organ) representative of all involved organs should be identified as target lesions and will be assessed at baseline and specified time points throughout the study. Tumor response for target lesions will be evaluated according to the RECIST 1.1:
Complete Response (CR): disappearance of all target lesions.
Partial Response (PR): at least 30% decrease from baseline in the sum of the longest diameters (longest axis for non-nodal lesions, short axis for nodal regions) of target lesions.
Progressive Disease (PD): at least 20% increase in the sum of the longest diameters (longest axis for non-nodal lesions, short axis for nodal regions) of target lesions, taking as a reference the smallest sum on study.
Stable Disease (SD): small changes that do not qualify for the above criteria

All other lesions (or sites of disease) should be identified as non-target lesions and will also be assessed at baseline and specified time points throughout the study. Tumor response for the group of non-target lesions will be evaluated according to the RECIST 1.1:
CR: Disappearance of all non-target lesions and normalization of CA15-3 tumor marker level. All lymph nodes must be non-pathological in size (<10 mm short axis).
Non-CR/Non-PD: Persistence of one or more non-target lesion(s) and/or maintenance of CA15-3 tumor marker level above the normal limits.
PD: Unequivocal progression of existing non-target lesions.

At each protocol-specified time point, the overall response will be determined as shown below:

| | | New | Overall |
|---|---|---|---|
| Target lesions | Non-target lesions | lesions | response |
| CR | CR | No | CR |
| CR | Non-CR/ non-PD | No | PR |
| CR | Not evaluated | No | PR |
| PR | Non-PD or not all | No | PR |
| | evaluated | | |
| SD | Non-PD or not all | No | SD |
| | evaluated | | |
| Not all | Non-PD | No | NE |
| evaluated | | | |
| PD | Any | Yes or No | PD |
| Any | PD | Yes or No | PD |
| Any | Any | Yes | PD |
| None (non- | CR | No | CR |
| measurable | Non-CR/non-PD | No | Non-CR/non- |
| disease) | | | PD |
| | Not all evaluated | No | NE |
| | Unequivocal PD | Yes or No | PD |
| | Any | Yes | PD |

1. A method of treating or ameliorating a cancer in a subject with one or more of a low monocyte count, a Luminal B breast cancer, an age of less than about 85 years, has been previously treated with a CDK4/6 inhibitor, has not previously undergone treatment with a taxane chemotherapy, has an elevated neutrophil to lymphocyte ratio, and diagnosed less than about 5 years ago, the method comprising administering to the subject an effective amount of a LAG-3 protein, or a derivative thereof that is able to bind to MHC class II molecules.

2. (canceled)

3. The method according to claim 1, wherein the cancer is a breast cancer.

4. The method according to claim 1, wherein the cancer is a hormone receptor-positive breast cancer.

5. The method according to claim 1, wherein the cancer is a hormone receptor-positive HER2 negative breast cancer.

6. The method according to claim 1, wherein the cancer is metastatic breast cancer.

7. The method according to claim 1, wherein the LAG-3 protein, or derivative thereof, is to be administered before, with, or after administration of a chemotherapy agent.

8. The method according to claim 7, wherein the LAG-3 protein, or derivative thereof, is to be administered after administration of the chemotherapy agent.

9. The method according to claim 7, wherein the chemotherapy agent is a taxane.

10. The method according to claim 9, wherein the taxane is paclitaxel.

11. The method according to claim 1, wherein the subject has one or more of a low monocyte count, has not previously undergone treatment with a taxane chemotherapy, has an elevated neutrophil to lymphocyte ratio, and was diagnosed less than about 5 years ago.

12. The method according to claim 1, wherein the subject has not previously undergone treatment with a taxane chemotherapy and has an elevated neutrophil to lymphocyte ratio.

13. The method according to claim 1, wherein the derivative of LAG-3 comprises:
the 30 amino acid extra-loop sequence GPPAAAPGHPLAPGPHPAAPSSWGPRPRRY (SEQ ID NO:2) of domain D1 of human LAG-3 protein; or
a variant of the 30 amino acid extra-loop sequence GPPAAAPGHPLAPGPHPAAPSSWGPRPRRY (SEQ ID NO:2) of domain D1 of human LAG-3 protein, wherein the variant comprises one or more amino acid substitutions, and has at least 70% amino acid identity with the 30 amino acid extra-loop sequence.

14. The method according to claim 1, wherein the derivative of LAG-3 protein comprises an amino acid sequence that has at least 70% amino acid identity with domain D1, and optionally domain D2, of LAG-3 protein, or at least 70% amino acid identity with domains D1, D2, D3, and optionally D4, of LAG-3 protein.

15. The method according to claim 1, wherein the derivative of LAG-3 protein is fused to Immunoglobulin Fc sequence.

16. The method according to claim 1, wherein the derivative of LAG-3 protein is IMP321.

17.-49. (canceled)


SEQUENCE LISTING (2 sequences)

SEQ ID NO: 1 - protein, 502 aa, Homo sapiens
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLTLGVLSLLLLVTGAFGFHLWRRQWRPRRFSALEQGIHPQAQSKIEELEQEPEPEPEPEPEPEPEPEPEQL

SEQ ID NO: 2 - protein, 30 aa, Homo sapiens
GPPAAAPGHPLAPGPHPAAPSSWGPRPRRY